(19) United States
(12) Patent Application Publication
(10) Pub. No.: US 2013/0195896 A1
(43) Pub. Date: Aug. 1, 2013

(54) COMPOSITIONS AND METHODS FOR THE TREATMENT OF A NEOPLASIA

(71) Applicant: David A. Goukassian, West Newton, MA (US)

(72) Inventor: David A. Goukassian, West Newton, MA (US)

(73) Assignee: STEWARD RESEARCH AND SPECIALTY PROJECTS CORPORATION, Boston, MA (US)

(21) Appl. No.: 13/639,815

(22) PCT Filed: Apr. 7, 2011
(86) PCT No.: PCT/US2011/031589

§ 371 Date: Apr. 11, 2013

Related U.S. Application Data
(60) Provisional application No. 61/321,657, filed on Apr. 7, 2010.

(51) Int. Cl. A61K 39/395; A61K 31/713

(52) U.S. Cl. CPC A61K 39/39558; A61K 31/713
USPC 424/174.1; 435/375; 514/44 A; 536/24.5

(57) ABSTRACT

The invention provides methods for the treatments of neoplasia featuring agents that interfere with the expression or activity of a TNFa receptor.

### RELATED APPLICATION

This application is related to U.S. provisional application Ser. Nos. 61/321,657, filed Apr. 7, 2010; 61/167,360, filed Apr. 7, 2009; 61/063,193, filed Jan. 31, 2008; and 61/123,096 filed Apr. 4, 2008, the entire contents of which are incorporated herein by this reference.

### BACKGROUND OF THE INVENTION

Cancer is a leading healthcare concern and a major leading cause of death worldwide. Recent studies have shown a link between inflammation and cancer. Tumor necrosis factor alpha (TNF-α) is a major mediator of inflammation and plays paradoxical roles in the progression of cancer. TNF-α, a 17 kDa polypeptide, induces many immunologically relevant and angiogenic genes through its interaction with two distinct receptors, TNF-αR1 (p55 receptor) and TNF-αcR2 (p75 receptor). Depending on TNF-α concentration and duration of exposure, TNF-α can act as either a tumor necrosis factor or a tumor-promoting factor. Endogenous TNF-α, which is continuously and chronically produced in tumors and the tumor microenvironment, promotes tumor growth, survival, differentiation, invasion, metastasis, and secretion of cytokines/chemokines, pro-angiogenic factors, and matrix metalloproteinases which are involved in cancer progression. In contrast, TNF-α administered locally at high-dose, is anti-angiogenic and pro-apoptotic, and displays a potent anti-tumor effect. Ongoing studies and results have shown the direct role of TNF-α receptors in tumor growth and cancer propagation. However, administration of TNF-α also induces systemic toxicity. Thus, a need exists for effective treatments of cancer utilizing the divergent roles of TNF-α in neoplastic survival and proliferation.

### SUMMARY OF THE INVENTION

As described below, the present invention features compositions and methods for inhibiting the expression or activity of one of two TNF-α receptors (p55 or p75).

In one aspect, the invention provides a method of reducing neoplastic cell survival or proliferation, the method involving contacting a neoplastic cell with an agent that selectively reduces the expression or activity of a p75 or p55 TNF-α receptor in the neoplastic cell relative to an untreated control cell, thereby reducing neoplastic cell survival or proliferation.

In another aspect, the invention provides a method of inhibiting angiogenesis in a neoplasia, the method involving contacting a neoplastic or endothelial cell with an agent that selectively reduces the expression or activity of a p55 or p75 TNF-α receptor in the neoplastic or endothelial cell relative to a reference, thereby inhibiting angiogenesis in the neoplasia.

In still another aspect, the invention provides a method of increasing cell death in a neoplasia, the method involving contacting a neoplastic cell with an agent that selectively reduces the expression or activity of a p55 or p75 TNF-α receptor in the neoplastic cell, thereby increasing cell death in the neoplasia, relative to an untreated control cell.

In yet another aspect, the invention provides a method of treating a subject with a neoplasia, the method involving administering to the subject an effective dose of an agent that selectively reduces the expression or activity of a p55 or p75 TNF-α receptor in a cell, thereby treating the subject with the neoplasia.

In yet another aspect, the invention provides a pharmaceutical composition for the treatment of a neoplasia, the composition containing an effective amount of an agent that selectively reduces the expression or activity of a p75 or p55 TNF-α receptor in a neoplastic cell, relative to a reference cell.

In yet another aspect, the invention provides a kit for treating neoplasia containing an effective amount of an agent that selectively reduces the expression or activity of a p75 or p55 TNF-α receptor in a neoplastic cell and instructions for using the kit to treat neoplasia.

In various embodiments of any of the above aspects, the method or composition selectively reduces the expression or activity of the p75 TNF-α receptor in the cell while the expression or activity of the p55 TNF-α receptor is not disrupted. In various embodiments of any of the above aspects, the method or composition selectively reduces the expression or activity of the p55 TNF-α receptor in the cell while the expression or activity of the p75 TNF-α receptor is not disrupted. In various embodiments of any of the above aspects, the method or composition selectively reduces expression by at least about 10% relative to the expression of the p75 or p55 TNF-α receptor in an untreated control cell. In various embodiments of any of the above aspects, the method or composition increases cell death or reduces blood vessel formation in a neoplasia or increases cell death in a neoplastic cell.

In various embodiments of any of the above aspects, the agent is an inhibitory nucleic acid molecule that is complementary to at least a portion of a p75 or p55 TNF-α receptor nucleic acid molecule. In various embodiments of any of the above aspects, the inhibitory nucleic acid molecule is any one or more of an antisense molecule, an siRNA, and an shRNA. In other embodiments of the invention, the inhibitory nucleic acid molecule contains a nucleic acid molecule with a sequence that is any one or more of SEQ ID NO: 1, SEQ ID NO: 2, SEQ ID NO: 3, and SEQ ID NO: 4. In other embodiments, the composition contains at least two inhibitory nucleic acid molecules. In a specific embodiment, the composition contains SEQ ID NOs: 1 and 2 or SEQ ID NOs: 1, 2, 3, and 4.

In various embodiments of any of the above aspects, the agent is an antibody or fragment thereof that selectively binds to the p75 or p55 TNF-α receptor. In other embodiments of the invention, the antibody is a monoclonal or polyclonal antibody.

In various embodiments of any of the above aspects, the agent is administered to a neoplastic cell or tumor in combination with tumor necrosis factor alpha (TNF-α). In particular embodiments, the TNF-α is administered intratumorally (e.g., by injection). In certain embodiments, the TNF-α is administered at low dose (0.5, 1, 2, 3, 4, 5, 6, 7, 8, 9, 10, 11, 12, 12.5, 13, 14, 15, or 20 μg/kg).

The invention provides compositions and methods for the treatment of neoplasia. Other features and advantages of the invention will be apparent from the detailed description, and from the claims.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “blood vessel formation” is meant the dynamic process that includes one or more steps of blood vessel development and/or maturation, such as angiogenesis, vasculogenesis, formation of an immature blood vessel network, blood vessel remodeling, blood vessel stabilization, blood vessel maturation, blood vessel differentiation, or establishment of a functional blood vessel network.

By “control” or “reference” is meant a standard of comparison. For example, the level of p75/TNF-α receptor in a neoplasia may be compared to the level of p75/TNF-αreceptor in a corresponding normal or healthy tissue.

By “inhibitory nucleic acid molecule” is meant a polynucleotide that disrupts the expression of a target nucleic acid molecule or an encoded polypeptide. Exemplary inhibitory nucleic acid moelcules include, but are not limited to, shRNAs, siRNAs, antisense nucleic acid molecules, and analogs thereof.

By “neoplasia” is meant a disease that is caused by or results in inappropriately high levels of cell division, inappropriately low levels of cell death, or both.

The term “portion” is meant to refer to a part of a polypeptide or nucleic acid molecule. This part contains, preferably, at least 10%, 20%, 30%, 40%, 50%, 60%, 70%, 80%, or 90% of the entire length of the reference nucleic acid molecule or polypeptide. A portion may contain 10, 20, 30, 40, 50, 60, 70, 80, 90, or 100, 200, 300, 400, 500, 600, 700, 800, 900, or 1000 nucleotides or amino acids.

By “subject” is meant a mammal, including, but not limited to, a human or non-human mammal, such as a bovine, equine, canine, ovine, or feline.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

The term “reduce” or “increase” is meant to alter negatively or positively, respectively, by at least 5%. An alteration may be by 5%, 10%, 25%, 30%, 50%, 75%, or even by 100%.

By “selectively” is meant the ability to affect the activity or expression of a target molecule without affecting the activity or expression of a non-target molecule. For example, a p75/TNF-α receptor inhibitory nucleic acid molecule selectively reduces the levels of the p75/TNF-α receptor without directly targeting the p55/TNF-α receptor.

By “TNF-α” is meant a protein or fragment thereof having substantial identity to the amino acid sequence of TNF-αprovided at GenBank Accession No. NP_{—}000585 that promotes angiogenesis or has binding activity to p55/TNF-α receptor or p75/TNF-αreceptor.

By “p75/TNFR2 polypeptide” is meant a protein or fragment thereof having substantial identity to the amino acid sequence of p75/TNFR2 provided at GenBank Accession No. NP_{—}001057 that promotes angiogenesis or has TNF-α binding activity.

By “p75/TNFR2 nucleic acid molecule” is meant a polynucleotide that encodes a p75/TNFR2 polypeptide.

By “p55/TNFR1 polypeptide” is meant a protein or fragment thereof having substantial identity to the amino acid sequence of p55/TNFR1 provided at GenBank Accession No. NP_{—}001056 that promotes angiogenesis or has TNF-α binding activity.

By “p55/TNFR1 nucleic acid molecule” is meant a polynucleotide that encodes a p55/TNFR1 polypeptide.

By “p75/TNFR2 biological activity” is meant TNF binding activity, p75/TNFR2 trimerization, p75/TNFR2-mediated signal transduction, stem and/or progenitor cell survival and proliferation, mobilization of bone marrow derived progenitor cells, stimulation of antioxidative pathways, angiogenesis enhancing activity, or regulation of cell adhesion molecules such as E-selectin, VCAM-1, ICAM.

By “p55/TNFR1 biological activity” is meant TNF binding activity, p55/TNFR2 trimerization, or p55/TNFR1-mediated signal transduction, angiogenesis enhancing activity, induction of apoptosis, or mediaton of inflammation, such as leukocyte infiltration.

The term “polynucleotide” or “nucleic acid” as used herein designates mRNA, RNA, cRNA, cDNA or DNA.

By the terms “polypeptide”, “peptide” and “protein” are meant to be used interchangeably herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally occurring amino acid polymers.

### BRIEF DESCRIPTION OF THE DRAWINGS

FIGS. 1A-1C show that the absence of the signaling via one (either TNFR1/p55 or TNFR2/p75), but not both TNF-α receptors, inhibited tumor growth, when compared to wild-type mice. (A) Representative images of mice with flank tumors (upper panel) and bisected tumors (lower panel) of WT, p75KO and p55KO. (B) Graphic representation of tumor volumes inoculated into flanks of WT, p75KO and p55KO mice. The graphs represent pooled data from three independent studies. (C) Representative tumors from WT, p75KO, p55KO and p55-p75 double KO mice.

FIGS. 2A and 2B show that there were statistically significant decreases in CD31 (an endothelial cell (EC) marker; also known as PECAM-1), staining in tumors of p75KO and p55KO mice relative to wild-type mice. (A) Representative images of CD31 immunostaining-red (upper panel) and Topro-3 nuclear staining-blue (lower panel). (B) Quantification of capillary density (CD31 intensity red staining in FIG. 2A in tumor tissue of WT, p75KO and p55KO mice.

FIGS. 3A and 3B show that there was a significant increase in cells positive for Terminal deoxynucleotidyl Transferase Biotin-dUTP Nick End Labeling (TUNEL) in tumors of p75KO and p55KO mice. (A) Representative images of TUNEL staining (upper panel) and Topro-3 nuclear staining-blue (lower panel). (B) Quantification of apoptosis (TUNEL intensity green staining in FIG. 3A, upper panel) in tumor tissue of WT, p75KO and p55KO mice.

FIGS. 4A-4C: (A) Images of tumors bisected from WT mice, panels from left to right: TUNEL immunostaining (green staining), capillary staining (CD31-red staining), Topro-3 nuclear staining (blue staining), and merged images. (B) Images of tumors bisected from p75KO mice, panels from left to right: TUNEL immunostaining (green staining), capillary staining (CD31-red staining), Topro-3 nuclear staining (blue staining), and merged images. (C) Images of tumors bisected from p55KO mice, panels from left to right: TUNEL immunostaining (green staining), capillary staining (CD31-red staining), Topro-3 nuclear staining (blue staining), and merged images.

FIGS. 5A-5C show that TNF-α expression was higher in tumor tissue in all genotypes. (A) Representative images from immunohistology using primary rat anti-TNF-αantibodies and species specific TRITC-labeled secondary antibodies: TNF-αimmunostaining-red (left column), Topro-3 nuclear staining-blue (second column), merged images (third column) and higher magnification (630×) insets of merged images (far right column). (B) Representative images of skin samples harvested form the same animals in (A) at least 3-4 cm away from tumor area: TNF-α Immunostaining, Topro-3 and merged images of skin section are shown. (C) Quantification of TNF-α immunostaining (TNF-α intensity of red staining in FIGS. 5A and B) in tumor tissues and normal skin of WT, p75KO and p55KO mice.

FIGS. 6A-6B show that VEGF immunostaining was reduced in tumor tissue from p75KO and p55KO mice. (A) Representative images of VEGF immunostaining (red; left column), Topro-3 nuclear staining (blue; second column), Topro-3 staining (third column) and higher magnification (630×) insets of merged images (far right column). (B) Quantification of VEGF immunostaining (VEGF intensity of red staining in FIG. 6A left column) in tumor tissues from WT, p75KO and p55KO mice.

FIGS. 7A-7D show that there was a significant decrease in the incorporation of green fluorescent protein (GFP)-labeled BM derived cells into the functional vessels network for p75KO mice. (A) Representative images of recruitment of BM derived cells into the tumor (GFP positive cells) for all genotypes (first column) were observed with no staining required. Arrowheads in all genotypes indicate the number of GFP positive BM derived cells recruited into the tumor. (B) Representative images of BM derived cells becoming a part of the functional vessels. Red staining indicates functional vessels stained with BS-1 lectin (animals were perfused with BS-1 lectin 5-10 minutes before sacrifice, a method that stains vessels that are perfused with blood). Large arrows indicate double positive BM derived cells (most likely endothelial progenitor cells (EPC's)) incorporated into functional vessels (yellow staining). Small arrowheads indicate BM derived cells not incorporated into functional vessels (green staining). (C) Quantification of the recruitment of BM-derived, GFP positive cells into the tumor in FIG. 7A. (D) Quantification of recruited BM-derived EPC's (positive for GFP and BS-1 lectin) into the functional vessels in FIG. 7B.

FIGS. 8A-8D show that the absence of p55 or p75 TNFα receptors in the host tissue inhibited gene expression in pathways regulating tumor angiogenesis. (A-C) LLC Angiogenesis Pathway Gene Array Expression at 5 minute exposure time among different genotypes (WT, p75KO and p55KO) showing various genes of interest and the loading controls at 5 second exposure time. Black circles indicate no change in gene expression between genotypes when compared to WT. Blue circles indicate a decrease in gene expression while red circles indicate increase in gene expression between genotypes when compared to WT. (D) Functional grouping of genes in LLC Angiogenesis functional pathway gene array with the fold changes in p75KO and p55KO genotypes when compared to WT (N=2-3 animals per genotype). Blue values indicate number of fold decrease in gene expression while red values indicate number of folds increase in gene expression for each genotype when compared to WT. Arbitrary cut off thresholds for fold increase/decrease were assigned at 1.6.

FIGS. 9A-9D show that the absence of p55 or p75 TNFα receptors in the host tissue significantly down regulated expression of anti-apoptotic and pro-survival genes. (A-C) LLC Apoptosis Pathway Gene Array Expression at 5 minute exposure time among different genotypes (WT, p75KO and p55KO) showing various genes of interest and the loading controls at 5 second exposure time. Black circles indicate no change in gene expression between genotypes when compared to WT. Blue circles indicate a decrease in gene expression between genotypes when compared to WT. (D) Functional grouping of genes in LLC Apoptosis functional pathway gene array with the fold changes in p75KO and p55KO genotypes when compared to WT (N=2-3 animals per genotype). Blue values indicate number of fold decrease in gene expression for each genotype when compared to WT. Arbitrary cut off thresholds for fold increase/decrease were assigned at 1.6.

FIGS. 10A-10D show that Bcl2-11, an apoptosis and cell senescence marker, increased in LLC tumors harvested from p75KO and p55KO mice. (A-C) LLC Cancer Pathway Gene Array Expression at 2 minute exposure time among different genotypes (WT, p75KO and p55KO) showing various genes of interest and the loading controls at 5 second exposure time. Black circles indicate no change in gene expression between genotypes when compared to WT. Blue circles indicate a decrease in gene expression between genotypes while red circles indicate an increase in gene expression between genotypes when compared to WT when compared to WT. (D) Functional grouping of genes in LLC Cancer pathway gene array with the fold changes in in p75KO and p55KO genotypes when compared to WT (N=2-3 animals per genotype). Blue values and minus sign indicate number of fold decrease in gene expression while red and plus sign indicate increase in gene expression for each genotype when compared to WT. Arbitrary cut off thresholds for fold increase/decrease were assigned at 1.6.

FIGS. 11A-11E show angiogenic gene expression analysed by qRT-PCR for Ang-1 (A), Ang-2 (B), MMP2 (C), Hif-1 alpha (D), and CXCL2 (E) in LLC tumor tissue from mice of WT, p75KO and p55KO genotype.

FIGS. 12A and 12B (A) Schematic of the plasmid backbone used in in vitro studies of p75R2 small hairpin (shRNA) trasfected LLC cells. (B) shRNA Sequences used in in vitro studies of p75R2 shRNA transfected LLC cells

FIGS. 13A and 13B show the results of in vitro studies of p75R2 shRNA trasfection in LLC cells. (A) Representative images of different combinations of shRNA plasmids transfected into LLCs cells and selected with geneticin (G418) for 6 days. (B) RT-PCR results plotted as a graph between RE (Relative Expression) and Samples (Plasmid Combinations) for TNFR2/p75 receptor expression in different TNFR2/p75 shRNA combonation transfected LLC cells.

FIGS. 14A and 14B show the effect of TNF treatment on LLCs transfected with various shRNAs. (A) FACS profiles of permanently transfected LLC cells with different combinations of TNFR2/p75 shRNA plasmids treated with TNF (80 ng/ml; high dose), harvested and stained with Propidium Iodide (PI) 24 hrs after TNF treatment. (B) Percent apoptotic LLCs (cells in sub-G0/G1 population).

FIGS. 15A-15C show the results of a qRT-PCR analysis of LLC tumors cells treated with TNF and processed for evaluation of angiogenic, pro-survival growth factor, and cytokine expression: (A) VEGF-A, HGF, and PLGF; (B) CXCR4, MMP2, Ezrin; and (C) Connexin43, Ang1, and Ang2.

FIGS. 16A-16D: (A) Chemotaxis and chemokinesis of WT and TNFR2/p75 EPCs in response to TNF-α (1 and 10 ng/ml; low doses), rmVEGF (20 ng/ml) and GM-CSF (50 ng/ml). (B) Examination of formation of tube-like structures in ex-vivo expanded WT and p75KO EPCs (5×10⁴ cells/well on 4-well chamber slides coated with Matrigel (Collaborative Biomedical Products, Bedford, Mass.) and incubated for 12 h in medium containing 5% FBS and supplemented with medium alone or 1 and/or 10 ng/ml of rmTNF-α (BD PharMingen)). Cells in the chambers were examined and photographs were taken 12 h post-stimulation. (C-D) mRNA expression of angiogenic factors in vitro from ex-vivo expanded WT and p75KO EPCs treated with 10 ng/ml of mrTNF-α. Ribonuclease Protection Assay (RPA) was performed using a custom made angiogenic factor (VEGF, bFGF and Angiopoeitin-1) multiprobe DNA template (PharMingen). [a ³²P]UTP (NEN) was used to synthesize in vitro transcribed antisense riboprobe and RPAs were carried out using RPA III TM kit (Ambion) following manufacturers instructions. Measurements are given in densitometric units.

FIGS. 17A-17C (A) Effect of TNF on NFκB nuclear translocation (NT) EPCs (grown in four well chamber slides) from WT and p75KO mice treated with TNF (10 ng/ml) for 30 minutes and processed for immunostaining with NFκB p65 (Santa Cruz Biotechnology). (B) Electromobility Shift Assay with NFκB consensus sequence to evaluate NFκB DNA binding activity. (C) Effect of TNF on NFκB-mediated VEGF promoter activity in EPCs from WT and p75KO mice transfected with full length (2.6 kb) VEGF promoter-reporter construct (in basic pGL2 plasmid backbone), a deletion construct (0.35 kb) containing two putative NFκB sites and one Sp1 cluster and an inactive deletion construct spanning up to −70 from transcription origination site and containing an incomplete Sp1 cluster.

FIG. 18 shows that the absence of the signaling via one (either TNFR1/p55 or TNFR2/p75), but not both TNFα receptors, inhibited aggressive B16 mouse melanoma growth. The graph shows tumor volumes of B16 melanomas inoculated into flanks of WT, p75KO and p55KO mice, representing pooled data from two independent studies. Representative images of bisected tumors (insets) of WT, p75KO and p55KO on day 14.

FIGS. 19A-19D shows that the absence of p55 or p75 TNFα receptors in the host tissue inhibited gene expression in pathways regulating tumor angiogenesis. (A-C) B16 mouse melanoma Angiogenesis Pathway Gene Array Expression at 5 minute exposure time between different genotypes (WT, p75KO and p55KO) showing various genes of interest and the loading controls at 5 second exposure time. Black circles indicate no change in gene expression between genotypes when compared to WT. Blue circles indicate decrease in gene expression while red circles indicate increase in gene expression between genotypes when compared to WT. (D) Functional grouping of genes in B16 Angiogenesis functional pathway gene array with the fold changes in p75KO and p55KO genotypes when compared to WT (N=2-3 animals per genotype). Blue values indicate number fold decrease in gene expression while red values indicate number of folds increase in gene expression for each genotype when compared to WT. Arbitrary cut off thresholds for fold increase/decrease were assigned at 1.6.

FIGS. 20A-20D show that the absence of p55 or p75 TNFα receptors in the host tissue affects the expression of anti-apoptotic and pro-survival genes. (A-C) B16 Apoptosis Pathway Gene Array Expression at 5 minute exposure time between different genotypes (WT, p75KO and p55KO) showing various genes of interest and the loading controls at 5 second exposure time. Black circles indicate no change in gene expression between genotypes when compared to WT. Blue circles indicate decrease in gene expression between genotypes when compared to WT. (D) Functional grouping of genes in B16 Apoptosis functional pathway gene array with the fold changes in p75KO and p55KO genotypes when compared to WT (N=2-3 animals per genotype). Blue values indicate number of folds decrease in gene expression for each genotype when compared to WT. Arbitrary cut off thresholds for fold increase/decrease were assigned at 1.6.

FIGS. 21A-21E show angiogenic gene expression analysed by qRT-PCR for Ang-1 (A), Hif-1alpha (B), MMP2 (C), Ang-2 (D), and CXCL2 (E) in B16 tumor tissue from mice of WT, p75KO and p55KO genotype.

FIG. 22 is a graph depicting mean number of p-γH2AX foci/cell in WT, p75KO, and p55KO EPC samples (non-irradiated control (CTRL) and 1Gy irradiation at various time points (24 hrs post media transfer from respective genotype CTRL and 1Gy irradiated EPCs). EPCs with 0 p-γH2AX foci/cells were excluded from the graph because in all three genotypes at any given time point 60-80% of EPCs had 0 p-γH2AX foci and no statistical difference was observed between genotypes at any time point.

FIG. 23 are graphs p-γH2AX foci distribution (>5 foci) in ex-vivo expanded BM-derived EPCs from WT, p55KO and p75KO mice treated with irradiated (1 Gy gamma) conditioned EPC medium from corresponding genotypes (WT, p55KO and p75KO). Please note: cells under normal conditions no-IR or IR medium transfer or any DNA damaging treatments may have 1-4 pH2AX foci. For clarity, the graphs are presented starting from cells with 5 foci. Panels (top-bottom) are graphs corresponding to control (CTRL), 5 hr, Day 1, Day 3, and Day 5 timepoints.

FIG. 24 are graphs depicting p-γH2AX foci distribution (1-4 foci) in ex-vivo expanded BM-derived EPCs from WT, p55KO and p75KO mice treated with irradiated (1 Gy gamma) conditioned EPC medium from corresponding genotypes (WT, p55KO and p75KO). Panels (top-bottom) are graphs corresponding to control (CTRL), 5 hr, Day 1, Day 3, and Day 5 timepoints.

FIGS. 25A and 25B are graphs showing inhibition of tumor growth when p75 levels are reduced in LLC tumors and inhibition of tumor growth when p75 levels are reduced and TNF is injected at low dose in LLC tumors. FIG. 22A is a graph showing that p75 knockout host microenvironment reduced growth (−60%) of unaltered LLCs (p75KO host+WT LLC). There was an additional decrease in tumor growth (−70%), when the microenvironment was p75KO and the LLCs were p75 receptor knockdown (KD) (p75KO host+p75 KD LLC). FIG. 22B is a graph showing that TNF injection increased survival (75%) of mice and inhibited tumor growth in p75 KD LLC tumors (WT host+p75 KD LLC; p75KO host+p75 KD LLC).

FIGS. 26A and 26B show sequences for a p75/TNFR2 polypeptide provided at GenBank Accession No. NP_{—}001057 (A); and for a p55/TNFR2 polypeptide provided at GenBank Accession No. NP_{—}001056 (B).

### DETAILED DESCRIPTION OF THE INVENTION

The invention features compositions and methods that are useful for the treatment or prevention of a neoplasia featuring compositions that inhibit the expression or activity of one of p75 or p55 TNF-α receptors, but not both simultaneously. In particular embodiments, the invention features compositions and methods that are useful for the treatment of a neoplasia by interfering with the expression or activity of either the TNF-α p75 receptor or the TNF-αp55 receptor. In some embodiments, when the expression or activity of one of a TNF-α p75 receptor or TNF-α p55 receptor is decreased, the activity of the TNF-α p55 or TNF-α p75 receptor, respectively, is increased (e.g., by contacting a neoplastic cell with TNF-α). The invention is based, at least in part, on the discovery that interfering with the expression of TNF-α p75 receptor or the TNF-α p55 receptor, but not interfering with the expression of both simultaneously, is useful for the treatment of neoplasia. As reported in more detail below, inhibiting the expression of either the TNF-α p75 receptor or the TNF-α p55 receptor, but not both simultaneously, reduced neoplastic cell growth and/or increased cell death in a neoplasia. Increased apoptosis and/or reduced angiogenesis in a neoplasia, or in an endothelial cell associated with a neoplasia, is observed when the expression of either the TNF-α p75 receptor or the TNF-α p55 receptor is inhibited. Accordingly, treatment with an agent that reduces the expression or biological activity of a TNF-α p75 receptor or a TNF-αp55 receptor is useful for reducing neoplastic cell survival or proliferation.

Accordingly, the invention provides methods of treating a neoplastic disease (e.g., lung cancer or melanoma) and/or disorders or symptoms thereof which comprise administering a therapeutically effective amount of a pharmaceutical composition comprising a compound (e.g., an inhibitory nucleic acid molecule that disrupts p75 or p55 TNF-αreceptor expression) described herein to a subject (e.g., a mammal such as a human). Thus, in one embodiment, the invention provides a method of treating a subject suffering from or susceptible to a neoplastic disease or disorder or symptom thereof. The method includes the step of administering to the mammal a therapeutic amount of a compound described herein sufficient to treat the disease or disorder or symptom thereof, under conditions such that the disease or disorder is treated.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of the compounds herein, such as a compound described herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, family history, and the like). The compounds herein may be also used in the treatment of any other disorders in which neoplasia, or an increased expression of p55 or p75 TNF-α receptors may be implicated.

### Tumor Necrosis Factor Alpha (TNF-α)

Tumor necrosis factor alpha (TNF-α) was first identified through the detection of antitumor activity in the sera of mice treated with endotoxin, and has been shown to be a member of a large family of related cytokines termed the TNF family. TNF family ligands are type II transmembrane proteins (intracellular N-terminus) that are biologically active as self assembling, non covalent bound, trimers. Some of these ligands, e.g. TNF-α, are active both as a membrane integrated form and as a soluble form released from the cell membrane after proteolytic cleavage, mainly by metalloproteinases induced by various stimuli.

Mature TNF-α is a 17 kDa polypeptide that specifically binds at least two cell surface receptors, p55 and p75 TNF-α receptors. These receptors have highly homologous extracellular domains, but show little homology between their intracellular domains. Without being tied to a particular theory, it is believed that p55 and p75 TNF-α receptors act through distinct signal transduction pathways. The p55 TNF-α receptor is generally responsible for signaling a variety of responses, including cytotoxicity and cytokine secretion, whereas the p75 TNF-α receptor is generally responsible for lymphoproliferative signals and the activation of T cells. TNF-α receptors are ubiquitously expressed on nearly all cell types, but the p75 TNF-α receptor is preferentially expressed by lymphoid cells, as well as other hemotopoeitic and endothelial cells.

Depending on TNF-α concentration and duration of exposure, TNF-α can act as either a tumor necrosis factor or a tumor-promoting factor. Endogenous TNF-α, which is constitutively produced in the tumor microenvironment, enhances tumor growth and induces cytokines/chemokines involved in cancer progression. Ongoing studies and results have shown the direct role of TNF-α receptors in tumor growth and cancer propagation. In contrast, TNF-α administered locally at high-dose, is anti-angiogenic and displays a potent anti-tumor effect. However, administration of TNF-α also induces systemic toxicity. Therefore, it is of interest to evaluate the consequences of TNF-α p75 receptor- and the TNF-α p55 receptor-deficiency on tumor survival and proliferation in TNF-α p75 receptor- and TNF-α p55 receptor-null mice. As reported herein, disruption of either the TNF-α p75 receptor or the TNF-α p55 receptor, but not both simultaneously, is associated with a reduction in neoplastic cell survival or proliferation. Increased apoptosis in neoplastic and endothelial cells and reduced angiogenesis in neoplasia are observed when the expression of either the TNF-α p75 receptor or the TNF-α p55 receptor is inhibited. Accordingly, the invention provides methods that reduce the expression or biological activity of the TNF-αp75 receptor or the TNF-α p55 receptor for the treatment of neoplasia, such as lung cancer and other human cancers.

### Neoplastic Cell Growth and Apoptosis

Neoplastic cell growth is not subject to the same regulatory mechanisms that govern the growth or proliferation of normal cells. Accordingly, agents that reduce the survival of a neoplasia, or that increase cell death (e.g., apoptosis) in a neoplasia are useful for the treatment of a neoplastic disease. Such compounds include agents, including but not limited to inhibitory nucleic acid molecules (e.g., antisense nucleic acid molecules, RNAi, siRNA, shRNA), that interfere with the expression or activity of the TNF-α p75 receptor or the TNF-α p55 receptor. Cancer is an example of a neoplasia. Examples of cancers include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases.

Agents that reduce the growth or proliferation of a neoplasm are useful for the treatment of neoplasms. Methods of assaying cell growth and proliferation are known in the art. See, for example, Kittler et al. (Nature. 432 (7020):1036-40, 2004) and Miyamoto et al. (Nature 416(6883):865-9, 2002). Assays for cell proliferation generally involve the measurement of DNA synthesis during cell replication. In one embodiment, DNA synthesis is detected using labeled DNA precursors, such as ([³H]-Thymidine or 5-bromo-2*-deoxyuridine [BrdU], which are added to cells (or animals) and then the incorporation of these precursors into genomic DNA during the S phase of the cell cycle (replication) is detected (Ruefli-Brasse et al., Science 302(5650):1581-4, 2003; Gu et al., Science 302 (5644):445-9, 2003).

Candidate compounds that reduce the survival of a neoplastic cell are also useful as anti-neoplasm therapeutics. Assays for measuring cell viability are known in the art, and are described, for example, by Crouch et al. (J. Immunol. Meth. 160, 81-8); Kangas et al. (Med. Bio1.62, 338-43, 1984); Lundin et al., (Meth. Enzymol. 133, 27-42, 1986); Petty et al. (Comparison of J. Biolum. Chemilum. 10, 29-34, 1995); and Cree et al. (AntiCancer Drugs 6: 398-404, 1995). Cell viability can be assayed using a variety of methods, including MTT (3-(4,5-dimethylthiazolyl)-2,5-diphenyltetrazolium bromide) (Barltrop, Bioorg. & Med. Chem. Lett. 1: 611, 1991; Cory et al., Cancer Comm. 3, 207-12, 1991; Paull J. Heterocyclic Chem. 25, 911, 1988). Assays for cell viability are also available commercially. These assays include but are not limited to CELLTITER-GLO® Luminescent Cell Viability Assay (Promega), which uses luciferase technology to detect ATP and quantify the health or number of cells in culture, and the CellTiter-Glo® Luminescent Cell Viability Assay, which is a lactate dehyrodgenase (LDH) cytotoxicity assay (Promega).

Candidate compounds that increase neoplastic cell death (e.g., increase apoptosis) are also useful as anti-neoplasm therapeutics. Assays for measuring cell apoptosis are known to the skilled artisan. Apoptotic cells are characterized by characteristic morphological changes, including chromatin condensation, cell shrinkage and membrane blebbing, which can be clearly observed using light microscopy. The biochemical features of apoptosis include DNA fragmentation, protein cleavage at specific locations, increased mitochondrial membrane permeability, and the appearance of phosphatidylserine on the cell membrane surface. Assays for apoptosis are known in the art. Exemplary assays include TUNEL (Terminal deoxynucleotidyl Transferase Biotin-dUTP Nick End Labeling) assays, caspase activity (specifically caspase-3) assays, and assays for fas-ligand and annexin V. Commercially available products for detecting apoptosis include, for example, Apo-ONE® Homogeneous Caspase-3/7 Assay, FragEL TUNEL kit (ONCOGENE RESEARCH PRODUCTS, San Diego, Calif.), the ApoBrdU DNA Fragmentation Assay (BIOVISION, Mountain View, Calif.), and the Quick Apoptotic DNA Ladder Detection Kit (BIOVISION, Mountain View, Calif.).

### Angiogenesis

Angiogenesis and/or vasculogenesis have been shown to play a role in maintaining or promoting neoplastic cell growth. By “angiogenesis” is meant the growth of new blood vessels originating from existing blood vessels. Methods for measuring angiogenesis are standard, and are described, for example, in Jain et al. (Nat. Rev. Cancer 2: 266-276, 2002). Angiogenesis can be assayed by measuring the number of non-branching blood vessel segments (number of segments per unit area), the functional vascular density (total length of perfused blood vessel per unit area), the vessel diameter, or the vessel volume density (total of calculated blood vessel volume based on length and diameter of each segment per unit area). By “vasculogenesis” is meant the development of new blood vessels originating from stem cells, angioblasts, or other precursor cells, such as endothelial progenitor cells (EPCs). These stem cells can be recruited from bone marrow endogenously or implanted therapeutically. As described herein, agents that reduce angiogenesis in a neoplasm are useful for the treatment of neoplasms.

### Assaying Compounds and Extracts

The invention provides methods for treating neoplasia by inhibiting neoplastic cell growth or increasing neoplastic cell death (e.g., by increasing apoptosis). In one embodiment, the method reduces neoplastic cell survival or increases neoplastic cell death by reducing the expression or activity of a p55/TNF-α receptor or a p75/TNF-α receptor by at least about 10, 20, 30, 40, 50, 60, 70, 80, 90, or 100% relative to the expression of the corresponding p55/TNF-α receptor or p75/TNF-α receptor in an untreated control cell. In one embodiment, the method provides an inhibitory nucleic acid molecule (e.g., a siRNA, shRNA, or antisense nucleic acid molecule) that binds to or that is complementary to at least a portion of a p55/TNF-α receptor or p75/TNF-α receptor nucleic acid molecule. While the Examples described herein specifically discuss the use of shRNA technology, one skilled in the art understands that the methods of the invention are not so limited. Virtually any agent that reduces the expression or activity of the p55/TNF-α receptor or p75/TNF-α receptor without reducing the expression or activity of both receptors may be employed in the methods of the invention. In another embodiment, the method provides an antibody or fragment thereof that binds to a p55/TNF-α receptor or p75/TNF-α receptor.

A selective p55/TNF-α receptor or p75/TNF-α receptor antagonist is one that reduces the expression of a p55/TNF-α receptor or p75/TNF-α receptor. Potential selective p55/TNF-α receptor or p75/TNF-α receptor antagonists include organic molecules, peptides, peptide mimetics, polypeptides (e.g., dominant negative polypeptides or proteins), nucleic acid ligands, aptamers, and antibodies that bind to a p55/TNF-α receptor or a p75/TNF-α receptor and selectively inhibit its activity. As used herein a “dominant negative polypeptide or protein” refers to a polypeptide or protein that adversely affects the normal, wild-type gene product when expressed within the same cell. This situation can occur if the product can interact with the same elements as the wild-type product, but block some aspect of its function. For example, a dominant negative fragment of either the p55/TNF-α receptor or p75/TNF-α receptor may contain the extracellular domain, but lack intracellular sequences. Trimerization between dominant negative polypeptides of either the p55/TNF-α receptor or p75/TNF-α receptor would affect signalling through their respective pathways because the dominant negative polypeptides lack the intracellular sequences necessary for signal transduction.

Methods of assaying the expression of a polypeptide or polynucleotide of interest are known in the art, and include co-immunoprecipitation, Western blotting, flow cytometry, immunocytochemistry, binding to magnetic and/or p55/TNF-α receptor- or p75/TNF-αreceptor-specific antibody-coated beads, in situ hybridization, fluorescence in situ hybridization (FISH), ELISA, microarray analysis, RT-PCR, Northern blotting, or colorimetric assays, such as the Bradford Assay and Lowry Assay. Potential antagonists also include small molecules that bind to and reduce the activity of the p55/TNF-α receptor or p75/TNF-α receptor.

Such agents may be used, for example, as a therapeutic to combat neoplasia in a subject. Optionally, agents identified in any of the assays described herein may be confirmed as useful in conferring protection against the development of neoplasia in any standard animal model (e.g., LLC tumors in mice) and, if successful, may be used as anti-neoplasia therapeutics.

Agents that selectively reduce the expression or activity of the p55/TNF-α receptor or p75/TNF-α receptor are identified as useful in the methods of the invention. In one embodiment, an agent that reduces the expression or activity of the p55/TNF-α receptor or p75/TNF-α receptor, is isolated and tested for its activity on neoplastic cell growth or survival in an in vitro assay. In another embodiment, the candidate agent is tested in an in vitro assay for its ability to reduce the expression or activity of one, but not both receptors. Additionally or optionally, the candidate agent is tested in an in vivo assay for its ability to inhibit neoplastic proliferation or increase neoplastic cell death. One skilled in the art appreciates that the effects of a candidate agent on a cell is typically compared to a corresponding control cell not contacted with the candidate agent.

In one working example, one or more candidate agents are added at varying concentrations to culture medium containing a neoplastic cell. An agent that suppresses the expression of a p55/TNF-α receptor or p75/TNF-α receptor expressed in the cell but does not suppress the expression of both receptors is considered useful in the invention; such an agent may be used, for example, as a therapeutic to prevent, delay, ameliorate, stabilize, or treat a neoplasia. Once identified, agents of the invention (e.g., agents that reduce the expression or activity of the p55/TNF-α receptor or p75/TNF-α receptor, but not both receptors) may be used to reduce neoplastic cell proliferation or increase neoplastic cell death in a patient in need thereof. Alternatively, an agent identified as useful to a method of the invention is locally or systemically delivered to decrease neoplastic cell proliferation or increase neoplastic cell death in situ.

In one example, a candidate compound that binds to a p55/TNF-α receptor or p75/TNF-α receptor may be identified using a chromatography-based technique. For example, a recombinant a p55/TNF-α receptor or p75/TNF-α receptor polypeptide of the invention may be purified by standard techniques from cells engineered to express the polypeptide, or may be chemically synthesized, once purified the peptide is immobilized on a column. A solution of candidate agents is then passed through the column, and an agent that specifically binds the p55/TNF-α receptor or p75/TNF-α receptor polypeptide or a fragment thereof is identified on the basis of its ability to bind to the p55/TNF-α receptor or p75/TNF-αreceptor polypeptide and to be immobilized on the column. To isolate the agent, the column is washed to remove non-specifically bound molecules, and the agent of interest is then released from the column and collected. Agents isolated by this method (or any other appropriate method) may, if desired, be further purified (e.g., by high performance liquid chromatography). In addition, these candidate agents may be tested for their ability to inhibit neoplastic cell proliferation (e.g., as described herein). Agents isolated by this approach may also be used, for example, as therapeutics to treat or prevent neoplasia (e.g., cancer). Compounds that are identified as reducing the expression or activity of the p55/TNF-αreceptor or p75/TNF-α receptor at least 10, 20, 30, 40, 50, 60, 70, 80, 90, or 100% are considered particularly useful in the invention.

Such agents may be used, for example, as a therapeutic to combat neoplasia in a subject. Optionally, agents identified in any of the above-described assays may be confirmed as useful in conferring protection against the development of neoplasia in any standard animal model (e.g., LLC tumors in mice) and, if successful, may be used as anti-neoplasia therapeutics.

In general, p55/TNF-α receptor or p75/TNF-α receptor antagonists (e.g., agents that selectively reduce the expression or activity of either p55/TNF-α receptor or p75/TNF-αreceptor, but not both) can be identified from large libraries of natural product or synthetic (or semi-synthetic) extracts or chemical libraries or from polypeptide or nucleic acid libraries, according to methods known in the art. Those skilled in the field of drug discovery and development will understand that the precise source of test extracts or compounds is not critical to the screening procedure(s) of the invention. Agents used in screens may include those known as therapeutics for the treatment of neoplasia. Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as the modification of existing polypeptides.

Libraries of natural polypeptides in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). Such polypeptides can be modified to include a protein transduction domain using methods known in the art and described herein. In addition, natural and synthetically produced libraries are produced, if desired, according to methods known in the art, e.g., by standard extraction and fractionation methods. Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994. Furthermore, if desired, any library or compound is readily modified using standard chemical, physical, or biochemical methods.

Numerous methods are also available for generating random or directed synthesis (e.g., semi-synthesis or total synthesis) of any number of polypeptides, chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, chemical compounds to be used as candidate compounds can be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Nall Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:6378-6382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity should be employed whenever possible.

When a crude extract is found to have p55/TNF-α receptor- or p75/TNF-α receptor-inhibitory activity further fractionation of the positive lead extract is necessary to isolate molecular constituents responsible for the observed effect. Thus, the goal of the extraction, fractionation, and purification process is the careful characterization and identification of a chemical entity within the crude extract that reduces the expression or activity of the p55/TNF-α receptor or p75/TNF-α receptor, without reducing the expression or activity of the p75/TNF-α receptor or p55/TNF-α receptor, respectively. Methods of fractionation and purification of such heterogenous extracts are known in the art. Methods of assaying the expression or activity of the p55/TNF-α and p75/TNF-α receptors are known in the art and can be used to determine whether an agent is selective for a p55/TNF-α receptor or p75/TNF-α receptor. If desired, compounds shown to be useful as therapeutics are chemically modified according to methods known in the art, e.g., to provide selectivity between the p55/TNF-α and p75/TNF-α receptors

### Inhibitory Nucleic Acids

Inhibitory nucleic acid molecules are those oligonucleotides that selectively inhibit the expression or activity of a p55 or p75 TNF-α receptor polypeptide or nucleic acid molecule. Such oligonucleotides include single and double stranded nucleic acid molecules (e.g., DNA, RNA, and analogs thereof) that are complementary to or that bind a nucleic acid molecule that encodes a TNF-α receptor polypeptide (e.g., antisense molecules, RNAi, siRNA, shRNA) as well as nucleic acid molecules that bind directly to a p55 or p75 TNF-αreceptor polypeptide to modulate its biological activity (e.g., aptamers).

siRNA

Short twenty-one to twenty-five nucleotide double-stranded RNAs are effective at down-regulating gene expression (Zamore et al., Cell 101: 25-33; Elbashir et al., Nature 411: 494-498, 2001, hereby incorporated by reference). The therapeutic effectiveness of an siRNA approach in mammals was demonstrated in vivo by McCaffrey et al. (Nature 418: 38-39.2002).

Given the sequence of a target gene, siRNAs may be designed to inactivate that gene. Such siRNAs, for example, could be administered directly to an affected tissue, or administered systemically. The nucleic acid sequence of a p55 or p75 TNF-α receptor polypeptide gene can be used to design small interfering RNAs (siRNAs). The 21 to 25 nucleotide siRNAs may be used, for example, as therapeutics to treat a neoplastic disease or disorder.

The inhibitory nucleic acid molecules of the present invention may be employed as double-stranded RNAs for RNA interference (RNAi)-mediated knock-down of p55 or p75 TNF-α receptor polypeptide expression. In one embodiment, p55 or p75 TNF-α receptor polypeptide expression is reduced in a neoplastic cell or an endothelial cell. RNAi is a method for decreasing the cellular expression of specific proteins of interest (reviewed in Tuschl, Chembiochem 2:239-245, 2001; Sharp, Genes & Devel. 15:485-490, 2000; Hutvagner and Zamore, Curr. Opin. Genet. Devel. 12:225-232, 2002; and Hannon, Nature 418:244-251, 2002). The introduction of siRNAs into cells either by transfection of dsRNAs or through expression of siRNAs using a plasmid-based expression system is increasingly being used to create loss-of-function phenotypes in mammalian cells.

### Ribozymes

Catalytic RNA molecules or ribozymes that include an antisense p55 or p75 TNF-αreceptor sequence of the present invention can be used to inhibit expression of a p55 or p75 TNF-α receptor nucleic acid molecule or polypeptide in vivo. The inclusion of ribozyme sequences within antisense RNAs confers RNA-cleaving activity upon them, thereby increasing the activity of the constructs. The design and use of target RNA-specific ribozymes is described in Haseloff et al., Nature 334:585-591. 1988, and U.S. Patent Application Publication No. 2003/0003469 A1, each of which is incorporated by reference.

Accordingly, the invention also features a catalytic RNA molecule that includes, in the binding arm, an antisense RNA having between eight and nineteen consecutive nucleobases. In preferred embodiments of this invention, the catalytic nucleic acid molecule is formed in a hammerhead or hairpin motif. Examples of such hammerhead motifs are described by Rossi et al., Aids Research and Human Retroviruses, 8:183, 1992. Example of hairpin motifs are described by Hampel et al., “RNA Catalyst for Cleaving Specific RNA Sequences,” filed Sep. 20, 1989, which is a continuation-in-part of U.S. Ser. No. 07/247,100 filed Sep. 20, 1988, Hampel and Tritz, Biochemistry, 28:4929, 1989, and Hampel et al., Nucleic Acids Research, 18: 299, 1990. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule.

shRNA

Small hairpin RNAs consist of a stem-loop structure with optional 3′ UU-overhangs. While there may be variation, stems can range from 21 to 31 bp (desirably 25 to 29 bp), and the loops can range from 4 to 30 bp (desirably 4 to 23 bp). In one embodiment of the invention, the shRNA molecule is made that includes between eight and twenty-one consecutive nucleobases of a p55 or p75 TNF-α receptor gene. In specific embodiments, the shRNA comprises a sequence represented by SEQ ID NO: 1, SEQ ID NO: 2, SEQ ID NO: 3, or SEQ ID NO: 4. For expression of shRNAs within cells, plasmid vectors containing either the polymerase III H1-RNA or U6 promoter, a cloning site for the stem-looped RNA insert, and a 4-5-thymidine transcription termination signal can be employed (e.g., pGeneClip Neomycin Vector; Promega Corporation). The Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs.

For expression of shRNAs within cells, plasmid vectors containing either the polymerase III H1-RNA or U6 promoter, a cloning site for the stem-looped RNA insert, and a 4-5-thymidine transcription termination signal can be employed. The Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs. Additional methods for expressing the shRNA in mammalian cells are described in the references cited above.

### Oligonucleotides and Other Nucleobase Oligomers

At least two types of oligonucleotides induce the cleavage of RNA by RNase H: polydeoxynucleotides with phosphodiester (PO) or phosphorothioate (PS) linkages. Although 2′-OMe-RNA sequences exhibit a high affinity for RNA targets, these sequences are not substrates for RNase H. A desirable oligonucleotide is one based on 2′-modified oligonucleotides containing oligodeoxynucleotide gaps with some or all internucleotide linkages modified to phosphorothioates for nuclease resistance. The presence of methylphosphonate modifications increases the affinity of the oligonucleotide for its target RNA and thus reduces the IC₅₀. This modification also increases the nuclease resistance of the modified oligonucleotide. It is understood that the methods and reagents of the present invention may be used in conjunction with any technologies that may be developed, including covalently-closed multiple antisense (CMAS) oligonucleotides (Moon et al., Biochem J. 346:295-303, 2000; PCT Publication No. WO 00/61595), ribbon-type antisense (RiAS) oligonucleotides (Moon et al., J. Biol. Chem. 275:4647-4653, 2000; PCT Publication No. WO 00/61595), and large circular antisense oligonucleotides (U.S. Patent Application Publication No. US 2002/0168631 A1).

As is known in the art, a nucleoside is a nucleobase-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric structure can be further joined to form a circular structure; open linear structures are generally preferred. Within the oligonucleotide structure, the phosphate groups are commonly referred to as forming the backbone of the oligonucleotide. The normal linkage or backbone of RNA and DNA is a 3′ to 5′ phosphodiester linkage.

Specific examples of preferred nucleobase oligomers useful in this invention include oligonucleotides containing modified backbones or non-natural internucleo side linkages. As defined in this specification, nucleobase oligomers having modified backbones include those that retain a phosphorus atom in the backbone and those that do not have a phosphorus atom in the backbone. For the purposes of this specification, modified oligonucleotides that do not have a phosphorus atom in their internucleoside backbone are also considered to be nucleobase oligomers.

Nucleobase oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriest-ers, and boranophosphates having normal 3′-5′ linkages, 2′-5′ linked analogs of these, and those having inverted polarity, wherein the adjacent pairs of nucleoside units are linked 3′-5′ to 5′-3′ or 2′-5′ to 5′-2′. Various salts, mixed salts and free acid forms are also included. Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Nucleobase oligomers having modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative United States patents that teach the preparation of the above oligonucleotides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

In other nucleobase oligomers, both the sugar and the internucleoside linkage, i.e., the backbone, are replaced with novel groups. The nucleobase units are maintained for hybridization with a nucleic acid molecule encoding a p75/TNF-α receptor or p55/TNF-αreceptor. One such nucleobase oligomer, is referred to as a Peptide Nucleic Acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Methods for making and using these nucleobase oligomers are described, for example, in “Peptide Nucleic Acids: Protocols and Applications” Ed. P. E. Nielsen, Horizon Press, Norfolk, United Kingdom, 1999. Representative United States patents that teach the preparation of PNAs include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

In particular embodiments of the invention, the nucleobase oligomers have phosphorothioate backbones and nucleosides with heteroatom backbones, and in particular —CH₂₋NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂—, and —O—N(CH₃)—CH₂—CH₂—. In other embodiments, the oligonucleotides have morpholino backbone structures described in U.S. Pat. No. 5,034,506.

Nucleobase oligomers may also contain one or more substituted sugar moieties. Nucleobase oligomers comprise one of the following at the 2′ position: OH; F; O—, S—, or N-alkyl; O—, S—, or N-alkenyl; O—, S- or N-alkynyl; or O-alkyl-O-alkyl, wherein the alkyl, alkenyl, and alkynyl may be substituted or unsubstituted C₁ to C₁₀ alkyl or C₂ to C₁₀ alkenyl and alkynyl. Particularly preferred are O[(CH₂)ₙO]ₙCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred nucleobase oligomers include one of the following at the 2′ position: C₁ to C₁₀ lower alkyl, substituted lower alkyl, alkaryl, aralkyl, O-alkaryl, or O-aralkyl, SH, SCH₃, OCN, Cl, Br, CN, CF₃, OCF₃, SOCH₃, SO₂CH₃, ONO₂, NO₂, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of a nucleobase oligomer, or a group for improving the pharmacodynamic properties of an nucleobase oligomer, and other substituents having similar properties. Preferred modifications are 2′-O-methyl and 2′-methoxyethoxy(2′-β-CH₂CH₂OCH₃, also known as 2′-O-(2-methoxyethyl) or 2′-MOE). Another desirable modification is 2′-dimethylaminooxyethoxy (i.e., O(CH₂)₂ON(CH₃)₂), also known as 2′-DMAOE. Other modifications include, 2′-aminopropoxy(2′-OCH₂CH₂CH₂NH₂) and 2′-fluoro (2′-F). Similar modifications may also be made at other positions on an oligonucleotide or other nucleobase oligomer, particularly the 3′ position of the sugar on the 3′ terminal nucleotide or in 2′-5′ linked oligonucleotides and the 5′ position of 5′ terminal nucleotide. Nucleobase oligomers may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

Nucleobase oligomers may also include nucleobase modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases, such as 5-methylcytosine (5-me-C), 5-hydroxymethyl cytosine, xanthine, hypoxanthine, 2-aminoadenine, 6-methyl and other alkyl derivatives of adenine and guanine; 2-propyl and other alkyl derivatives of adenine and guanine; 2-thiouracil, 2-thiothymine and 2-thiocytosine; 5-halouracil and cytosine; 5-propynyl uracil and cytosine; 6-azo uracil, cytosine and thymine; 5-uracil (pseudouracil); 4-thiouracil; 8-halo, 8-amino, 8-thiol, 8-thioalkyl, 8-hydroxyl and other 8-substituted adenines and guanines; 5-halo (e.g., 5-bromo), 5-trifluoromethyl and other 5-substituted uracils and cytosines; 7-methylguanine and 7-methyladenine; 8-azaguanine and 8-azaadenine; 7-deazaguanine and 7-deazaadenine; and 3-deazaguanine and 3-deazaadenine. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of an antisense oligonucleotide of the invention. These include 5-substituted pyrimidines, 6-azapyrimidines, and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2.degree. C. (Sanghvi, Y. S., Crooke, S. T. and Lebleu, B., eds., Antisense Research and Applications, CRC Press, Boca Raton, 1993, pp. 276-278) and are desirable base substitutions, even more particularly when combined with 2′-O-methoxyethyl or 2′-O-methyl sugar modifications. Representative United States patents that teach the preparation of certain of the above noted modified nucleobases as well as other modified nucleobases include U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; and 5,750,692, each of which is herein incorporated by reference.

Another modification of a nucleobase oligomer of the invention involves chemically linking to the nucleobase oligomer one or more moieties or conjugates that enhance the activity, cellular distribution, or cellular uptake of the oligonucleotide. Such moieties include but are not limited to lipid moieties such as a cholesterol moiety (Letsinger et al., Proc. Natl. Acad. Sci. USA, 86:6553-6556, 1989), cholic acid (Manoharan et al., Bioorg. Med. Chem. Let, 4:1053-1060, 1994), a thioether, e.g., hexyl-5-tritylthiol (Manoharan et al., Ann. N.Y. Acad. Sci., 660:306-309, 1992; Manoharan et al., Bioorg. Med. Chem. Let., 3:2765-2770, 1993), a thiocholesterol (Oberhauser et al., Nucl. Acids Res., 20:533-538: 1992), an aliphatic chain, e.g., dodecandiol or undecyl residues (Saison-Behmoaras et al., EMBO J., 10:1111-1118, 1991; Kabanov et al., FEBS Lett., 259:327-330, 1990; Svinarchuk et al., Biochimie, 75:49-54, 1993), a phospholipid, e.g., di-hexadecyl-rac-glycerol or triethylammonium 1,2-di-O-hexadecyl-rac-glycero-3-H-phosphonate (Manoharan et al., Tetrahedron Lett., 36:3651-3654, 1995; Shea et al., Nucl. Acids Res., 18:3777-3783, 1990), a polyamine or a polyethylene glycol chain (Manoharan et al., Nucleosides & Nucleotides, 14:969-973, 1995), or adamantane acetic acid (Manoharan et al., Tetrahedron Lett., 36:3651-3654, 1995), a palmityl moiety (Mishra et al., Biochim. Biophys. Acta, 1264:229-237, 1995), or an octadecylamine or hexylamino-carbonyl-oxycholesterol moiety (Crooke et al., J. Pharmacol. Exp. Ther., 277:923-937, 1996. Representative United States patents that teach the preparation of such nucleobase oligomer conjugates include U.S. Pat. Nos. 4,587,044; 4,605,735; 4,667,025; 4,762,779; 4,789,737; 4,824,941; 4,828,979; 4,835,263; 4,876,335; 4,904,582; 4,948,882; 4,958,013; 5,082,830; 5,109,124; 5,112,963; 5,118,802; 5,138,045; 5,214,136; 5,218,105; 5,245,022; 5,254,469; 5,258,506; 5,262,536; 5,272,250; 5,292,873; 5,317,098; 5,371,241, 5,391,723; 5,414,077; 5,416,203, 5,451,463; 5,486,603; 5,510,475; 5,512,439; 5,512,667; 5,514,785; 5,525,465; 5,541,313; 5,545,730; 5,552,538; 5,565,552; 5,567,810; 5,574,142; 5,578,717; 5,578,718; 5,580,731; 5,585,481; 5,587,371; 5,591,584; 5,595,726; 5,597,696; 5,599,923; 5,599,928; 5,608,046; and 5,688,941, each of which is herein incorporated by reference.

The present invention also includes nucleobase oligomers that are chimeric compounds. “Chimeric” nucleobase oligomers are nucleobase oligomers, particularly oligonucleotides, that contain two or more chemically distinct regions, each made up of at least one monomer unit, i.e., a nucleotide in the case of an oligonucleotide. These nucleobase oligomers typically contain at least one region where the nucleobase oligomer is modified to confer, upon the nucleobase oligomer, increased resistance to nuclease degradation, increased cellular uptake, and/or increased binding affinity for the target nucleic acid. An additional region of the nucleobase oligomer may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of nucleobase oligomer inhibition of gene expression. Consequently, comparable results can often be obtained with shorter nucleobase oligomers when chimeric nucleobase oligomers are used, compared to phosphorothioate deoxyoligonucleotides hybridizing to the same target region.

Chimeric nucleobase oligomers of the invention may be formed as composite structures of two or more nucleobase oligomers as described above. Such nucleobase oligomers, when oligonucleotides, have also been referred to in the art as hybrids or gapmers. Representative United States patents that teach the preparation of such hybrid structures include U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference in its entirety.

The nucleobase oligomers used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

The nucleobase oligomers of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

### Delivery of Nucleobase Oligomers

Naked inhibitory nucleic acid molecules, or analogs thereof, are capable of entering mammalian cells and inhibiting expression of a gene of interest. Nonetheless, it may be desirable to utilize a formulation that aids in the delivery of oligonucleotides or other nucleobase oligomers to cells (see, e.g., U.S. Pat. Nos. 5,656,611, 5,753,613, 5,785,992, 6,120,798, 6,221,959, 6,346,613, and 6,353,055, each of which is hereby incorporated by reference).

### Antibodies

Antibodies that selectively bind a p75/TNF-α receptor or p55/TNF-α receptor and inhibit its activity are useful in the methods of the invention. In one embodiment, selective binding of antibody to the p75/TNF-α receptor reduces p75/TNF-α receptor biological activity, respectively, e.g., as assayed by analyzing binding to TNF-α. In another embodiment, selective binding of antibody to the p55/TNF-α receptor reduces p55/TNF-αreceptor biological activity. Desirably, the selective binding of antibody to the p75/TNF-αreceptor does not disrupt binding of TNFα to the p55/TNF-α receptor or any other p55/TNF-αreceptor biological activity. Likewise, the selective binding of antibody to the p55/TNF-αreceptor does not disrupt TNFα binding to the p75/TNF-α receptor or any other p75/TNF-αreceptor biological activity. Methods of preparing antibodies are well known to those of ordinary skill in the science of immunology. As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fc fragment of intact antibody, clear more rapidly from the circulation, and may have less non-specific tissue binding of an intact antibody (Wahl et al., J. Nucl. Med. 24:316-325 (1983). The antibodies of the invention comprise whole native antibodies, bispecific antibodies; chimeric antibodies; Fab, Fab′, single chain V region fragments (scFv), fusion polypeptides, and unconventional antibodies.

In one embodiment, an antibody that binds a p75/TNF-α receptor or p55/TNF-αreceptor is monoclonal. Alternatively, the anti-p75/TNF-α receptor or p55/TNF-α receptor antibody is a polyclonal antibody. The preparation and use of polyclonal antibodies are known to the skilled artisan. The invention also encompasses hybrid antibodies, in which one pair of heavy and light chains is obtained from a first antibody, while the other pair of heavy and light chains is obtained from a different second antibody. Such hybrids may also be formed using humanized heavy and light chains. Such antibodies are often referred to as “chimeric” antibodies.

In general, intact antibodies are said to contain “Fc” and “Fab” regions. The Fc regions are involved in complement activation and are not involved in antigen binding. An antibody from which the Fc′ region has been enzymatically cleaved, or which has been produced without the Fc′ region, designated an “F(ab′)₂” fragment, retains both of the antigen binding sites of the intact antibody. Similarly, an antibody from which the Fc region has been enzymatically cleaved, or which has been produced without the Fc region, designated an “Fab′” fragment, retains one of the antigen binding sites of the intact antibody. Fab′ fragments consist of a covalently bound antibody light chain and a portion of the antibody heavy chain, denoted “Fd.” The Fd fragments are the major determinants of antibody specificity (a single Fd fragment may be associated with up to ten different light chains without altering antibody specificity). Isolated Fd fragments retain the ability to specifically bind to immunogenic epitopes.

Antibodies can be made by any of the methods known in the art utilizing a p75/TNF-αreceptor or p55/TNF-α receptor, or immunogenic fragments thereof, as an immunogen. One method of obtaining antibodies is to immunize suitable host animals with an immunogen and to follow standard procedures for polyclonal or monoclonal antibody production. The immunogen will facilitate presentation of the immunogen on the cell surface. Immunization of a suitable host can be carried out in a number of ways. Nucleic acid sequences encoding a p75/TNF-α receptor or p55/TNF-α receptor or immunogenic fragments thereof, can be provided to the host in a delivery vehicle that is taken up by immune cells of the host. The cells will in turn express the receptor on the cell surface generating an immunogenic response in the host. Alternatively, nucleic acid sequences encoding a p75/TNF-α receptor or p55/TNF-α receptor, or immunogenic fragments thereof, can be expressed in cells in vitro, followed by isolation of the polypeptide and administration of the polypeptide to a suitable host in which antibodies are raised.

Alternatively, antibodies against a p75/TNF-α receptor or p55/TNF-α receptor may, if desired, be derived from an antibody phage display library. A bacteriophage is capable of infecting and reproducing within bacteria, which can be engineered, when combined with human antibody genes, to display human antibody proteins. Phage display is the process by which the phage is made to ‘display’ the human antibody proteins on its surface. Genes from the human antibody gene libraries are inserted into a population of phage. Each phage carries the genes for a different antibody and thus displays a different antibody on its surface.

Antibodies made by any method known in the art can then be purified from the host. Antibody purification methods may include salt precipitation (for example, with ammonium sulfate), ion exchange chromatography (for example, on a cationic or anionic exchange column preferably run at neutral pH and eluted with step gradients of increasing ionic strength), gel filtration chromatography (including gel filtration HPLC), and chromatography on affinity resins such as protein A, protein G, hydroxyapatite, and anti-immunoglobulin.

Antibodies can be conveniently produced from hybridoma cells engineered to express the antibody. Methods of making hybridomas are well known in the art. The hybridoma cells can be cultured in a suitable medium, and spent medium can be used as an antibody source. Polynucleotides encoding the antibody of interest can in turn be obtained from the hybridoma that produces the antibody, and then the antibody may be produced synthetically or recombinantly from these DNA sequences. For the production of large amounts of antibody, it is generally more convenient to obtain an ascites fluid. The method of raising ascites generally comprises injecting hybridoma cells into an immunologically naive histocompatible or immunotolerant mammal, especially a mouse. The mammal may be primed for ascites production by prior administration of a suitable composition (e.g., Pristane).

Monoclonal antibodies (Mabs) produced by methods of the invention can be “humanized” by methods known in the art. “Humanized” antibodies are antibodies in which at least part of the sequence has been altered from its initial form to render it more like human immunoglobulins. Techniques to humanize antibodies are particularly useful when non-human animal (e.g., murine) antibodies are generated. Examples of methods for humanizing a murine antibody are provided in U.S. Pat. Nos. 4,816,567, 5,530,101, 5,225,539, 5,585,089, 5,693,762 and 5,859,205.

In other embodiments, the invention provides “unconventional antibodies.” Unconventional antibodies include, but are not limited to, nanobodies, linear antibodies (Zapata et al., Protein Eng. 8(10): 1057-1062, 1995), single domain antibodies, single chain antibodies, and antibodies having multiple valencies (e.g., diabodies, tribodies, tetrabodies, and pentabodies). Nanobodies are the smallest fragments of naturally occurring heavy-chain antibodies that have evolved to be fully functional in the absence of a light chain. Nanobodies have the affinity and specificity of conventional antibodies although they are only half of the size of a single chain Fv fragment. The consequence of this unique structure, combined with their extreme stability and a high degree of homology with human antibody frameworks, is that nanobodies can bind therapeutic targets not accessible to conventional antibodies. Recombinant antibody fragments with multiple valencies provide high binding avidity and unique targeting specificity to cancer cells. These multimeric scFvs (e.g., diabodies, tetrabodies) offer an improvement over the parent antibody since small molecules of ˜60-100 kDa in size provide faster blood clearance and rapid tissue uptake See Power et al., (Generation of recombinant multimeric antibody fragments for tumor diagnosis and therapy. Methods Mol Biol, 207, 335-50, 2003); and Wu et al. (Anti-carcinoembryonic antigen (CEA) diabody for rapid tumor targeting and imaging. Tumor Targeting, 4, 47-58, 1999).

Various techniques for making unconventional antibodies have been described. Bispecific antibodies produced using leucine zippers are described by Kostelny et al. (J. Immunol. 148(5):1547-1553, 1992). Diabody technology is described by Hollinger et al. (Proc. Natl. Acad. Sci. USA 90:6444-6448, 1993). Another strategy for making bispecific antibody fragments by the use of single-chain Fv (sFv) diners is described by Gruber et al. (J. Immunol. 152:5368, 1994). Trispecific antibodies are described by Tutt et al. (J. Immunol. 147:60, 1991). Single chain Fv polypeptide antibodies include a covalently linked VH::VL heterodimer which can be expressed from a nucleic acid including V_{H}- and V_{L}-encoding sequences either joined directly or joined by a peptide-encoding linker as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754.

### Therapy

Anti-neoplasia therapy employing a p55/TNFα receptor antagonist or a p75/TNFα receptor antagonist (e.g., an agent that inhibits the expression or activity of one such receptor) is also provided by the invention. Therapy may be provided wherever cancer therapy is performed: at home, the doctor's office, a clinic, a hospital's outpatient department, or a hospital. Treatment generally begins at a hospital so that the doctor can observe the therapy's effects closely and make any adjustments that are needed. The duration of the therapy depends on the kind of cancer being treated, the age and condition of the patient, the stage and type of the patient's disease, and how the patient's body responds to the treatment. Drug administration may be performed at different intervals (e.g., daily, weekly, or monthly). Therapy may be given in on-and-off cycles that include rest periods so that the patient's body has a chance to build healthy new cells and regain its strength.

Depending on the type of cancer and its stage of development, the therapy can be used to slow the spreading of the cancer, to slow the cancer's growth, to kill or arrest cancer cells that may have spread to other parts of the body from the original tumor, to relieve symptoms caused by the cancer, or to prevent cancer in the first place.

As used herein, the terms “cancer” or “neoplasm” or “neoplastic cells” is meant a collection of cells multiplying in an abnormal manner. Cancer growth is uncontrolled and progressive, and occurs under conditions that would not elicit, or would cause cessation of, multiplication of normal cells.

An inhibitory nucleic acid described herein, or other selective inhibitor of p75/TNF-αreceptor or p55/TNF-α receptor, may be administered within a pharmaceutically-acceptable diluent, carrier, or excipient, in unit dosage form. Conventional pharmaceutical practice may be employed to provide suitable formulations or compositions to administer the compounds to patients suffering from a disease that is caused by excessive cell proliferation. Administration may begin before the patient is symptomatic. Any appropriate route of administration may be employed, for example, administration may be topical, parenteral, intravenous, intraarterial, subcutaneous, intratumoral, intramuscular, intracranial, intraorbital, ophthalmic, intraventricular, intrahepatic, intracapsular, intrathecal, intracisternal, intraperitoneal, intranasal, aerosol, suppository, or oral administration. For example, therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found, for example, in “Remington: The Science and Practice of Pharmacy” Ed. A. R. Gennaro, Lippincourt Williams & Wilkins, Philadelphia, Pa., 2000. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for p75/TNF-α receptor- or p55/TNF-αreceptor-modulatory compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The formulations can be administered to human patients in therapeutically effective amounts (e.g., amounts which prevent, eliminate, or reduce a pathological condition) to provide therapy for a disease or condition. “Therapeutically effective amount” is intended to include an amount of a compound useful in the present invention or an amount of the combination of compounds claimed, e.g., to treat or prevent the disease or disorder, or to treat the symptoms of the disease or disorder, in a host. The combination of compounds is preferably a synergistic combination. Synergy, as described for example by Chou and Talalay, Adv. Enzyme Regul. 22:27-55 (1984), occurs when the effect of the compounds when administered in combination is greater than the additive effect of the compounds when administered alone as a single agent. In general, a synergistic effect is advantageously demonstrated at suboptimal concentrations of the compounds. Synergy can be in terms of lower cytotoxicity, increased activity, or some other beneficial effect of the combination compared with the individual components. The preferred dosage of an inhibitory nucleic acid of the invention is likely to depend on such variables as the type and extent of the disorder, the overall health status of the particular patient, the formulation of the compound excipients, and its route of administration.

For any of the methods of application described above, an agent of the invention is desirably administered intravenously or is applied to the site of the needed anti-proliferative or anti-neoplasia event (e.g., by injection).

For any of the methods of application described above, treatment with an agent of the invention may be combined with administration of TNF-α to a neoplastic cell or solid tumor (e.g., by injection). In certain embodiments, the TNF-α is administered at a low dose based on body weight (0.5, 1, 2, 3, 4, 5, 6, 7, 8, 9, 10, 11, 12, 12.5, 13, 14, 15, or 20 μg/kg). In the event that a response in a subject is insufficient at the initial doses applied, the TNF-α dose may be adjusted. In a particular embodiment, the TNF-α is administered intratumorally by injection.

As described above, if desired, treatment with an agent of the invention may be combined with therapies for the treatment of proliferative disease (e.g., radiotherapy, surgery, or chemotherapy).

### Tumors

The invention provides agents that selectively inhibit a p55 or p75 TNFα receptor in a subject having a neoplasia, thereby reducing neoplastic cell growth or survival. In one embodiment, an agent of the invention reduces blood vessel formation within the neoplasia, reduces neoplastic cell survival, or increases cell death in the neoplasia or in a blood vessel associated with the neoplasia. Reduction of tumor growth means a measurable decrease in growth of the tumor of at least about 0.01-fold (for example 0.01, 0.1, 1, 3, 4, 5, 10, 100, 1000-fold or more) or decrease by at least about 0.01% (for example 0.01, 0.1, 1, 3, 4, 5, 10, 20, 30, 40, 50, 60, 70, 80, 90, 95, 99 or 100%) as compared to the growth measured over time prior to treatment as defined herein.

Full eradication of the tumor may also be achieved through methods of the invention. Eradication refers to elimination of the tumor. The tumor is considered to be eliminated when it is no longer detectable using detection methods known in the art (e.g., imaging).

### Anti-Tumor Therapy

Contemplated herein as anti-tumor therapy administered to the subject being treated for a solid tumor according to a method of the invention (in addition to the agent or therapy effecting reduction in the expression or activity of a p75/TNF-α receptor or p55/TNF-αreceptor) are, without limitation, surgery, radiation, chemotherapy, cytotoxic agents, and immune activators. Such therapies can be used in combination with an agent or therapy effecting reduction in the expression or activity of a p75/TNF-α receptor or p55/TNF-αreceptor

Cytotoxic agents include chemotherapeutic agents, radiation therapy, and anti-angiogenic agents. The cytotoxic agent can be a chemical agent, such as a chemotherapeutic agent used in cancer treatment (adriamycin or etoposide, for example) or hormones such as tamoxifen or other biologicals such as TNF-α or bFGF. In one embodiment, the anti-angiogenic agent modulates a vascular endothelial growth factor receptor, such as vascular endothelial growth factor receptor-2, by blocking the receptor. For example, the anti-angiogenic agent can be an antibody, such as DC101, Avastin and Herceptin.

The anti-angiogenic agent can also be, but is not limited to, Endostatin, Angiostatin, Galardin (GM6001, Glycomed, Inc., Alameda, Calif.), low molecular weight VEGF receptor kinases (e.g., Novartis PTK787 and AstraZeneca ADZ2171), endothelial response inhibitors (e.g., agents such as interferon alpha, TNP470, and vascular endothelial growth factor inhibitors), agents that prompt the breakdown of the cellular matrix (e.g., Vitaxin (human LM-609 antibody, Ixsys Co., San Diego, Calif.; Metastat, CollaGenex, Newtown, Pa.; and Marimastat BB2516, British Biotech), agents that act directly on vessel growth (e.g., CM-101, which is derived from exotoxin of Group A Streptococcus antigen and binds to new blood vessels inducing an intense host inflammatory response; and Thalidomide), a synthetic progesterone (e.g., medroxyprogesterone acetate (MPA), Oikawa (1988) Cancer Lett. 43: 85), a pro-drug of 5FU (e.g., 5′-deoxy-5-fluorouridine (5′DFUR), Haraguchi (1993) Cancer Res. 53: 5680-5682; Yayoi (1994) Int J. Oncol. 5: 27-32; Yamamoto (1995) Oncol Reports 2:793-796), and polysaccharides capable of interfering with the function of heparin-binding growth factors that promote angiogenesis (e.g., pentosan polysulfate).

The “chemotherapeutic agent” includes chemical reagents that inhibit the growth of proliferating cells or tissues wherein the growth of such cells or tissues is undesirable. Chemotherapeutic agents are well known in the art (see e.g., Gilman A. G., et al., The Pharmacological Basis of Therapeutics, 8th Ed., Sec 12:1202-1263 (1990)), and Teicher, B. A. Cancer Therapeutics: Experimental and Clinical Agents (1996) Humana Press, Totowa, N.J. Other similar examples of chemotherapeutic agents include: bleomycin, docetaxel (Taxotere), doxorubicin, edatrexate, erlotinib (Tarceva), etoposide, finasteride (Proscar), flutamide (Eulexin), gemcitabine (Gemzar), genitinib (Irresa), goserelin acetate (Zoladex), granisetron (Kytril), imatinib (Gleevec), irinotecan (Campto/Camptosar), ondansetron (Zofran), paclitaxel (Taxol), pegaspargase (Oncaspar), pilocarpine hydrochloride (Salagen), porfimer sodium (Photofrin), interleukin-2 (Proleukin), rituximab (Rituxan), topotecan (Hycamtin), trastuzumab (Herceptin), tretinoin (Retin-A), Triapine, vincristine, and vinorelbine tartrate (Navelbine).

### Kits

The invention provides kits for the treatment or prevention of a neoplasia. In one embodiment, the kit includes a therapeutic or prophylactic composition containing an effective amount of an agent described herein, such as an inhibitory nucleic acid described herein (e.g., an shRNA comprising SEQ ID NO:1, SEQ ID NO:2, SEQ ID NO:3 or SEQ ID NO:4) in unit dosage form. In some embodiments, the kit comprises a sterile container that contains a therapeutic or prophylactic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired an agent of the invention is provided together with instructions for administering the agent to a subject having or at risk of developing a neoplasia. The instructions will generally include information about the use of the composition for the treatment or prevention of neoplasia. In other embodiments, the instructions include at least one of the following: description of the therapeutic agent; dosage schedule and administration for treatment or prevention of ischemia or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### EXAMPLES

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### Example 1

### Tumor Growth was Inhibited in p75KO Mice and p55KO Mice

To evaluate and understand the role of TNF-α in tumor growth, mouse models of tumorigenesis were used to compare the effect of a host microenvironment on the growth and development of tumors over time. Three sets of independent experiments were performed on mice with the following genotypes: wild-type (WT; C57BL/6), TNFR1/p55 knockout (p55KO), and TNFR2/p75KO knockout (p75KO). For each experiment, at least 6-8 animals were used for each mouse genotype. Mice (6-8 weeks old) were injected with cells of a lung adenocarcinoma cell line (Lewis lung carcinoma; LLC; 5×10⁵ cells of mouse lung in 50 μl of growth reduced Matrigel) into the flank. Before injection, the viability of LLCs was determined to be more than 95% (trypan blue exclusion method). Tumor growth in the mice was evaluated by measuring the length, width and height of tumors weekly using electronic calipers. Tumor volumes were calculated using standard mathematical formulas for volume (i.e., V=L×W×H or V=4/3πr³; similar volumes were obtained by either method). Data from the three independent studies were pooled for analysis. No mortality was observed in any of the mouse genotypes injected with LLCs up to day 21 post-inoculation when the experiments were terminated.

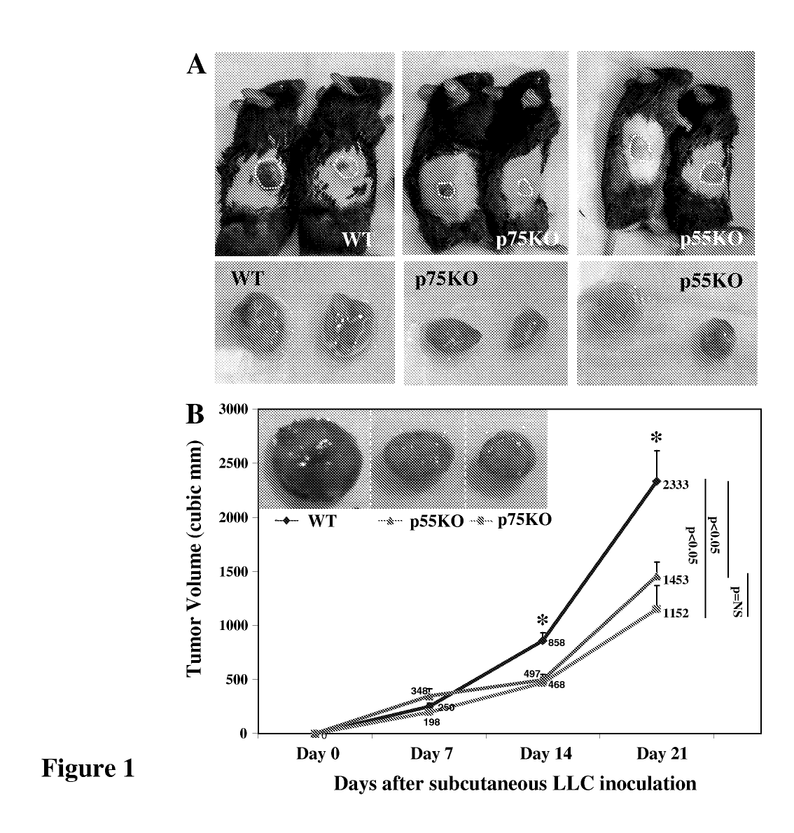

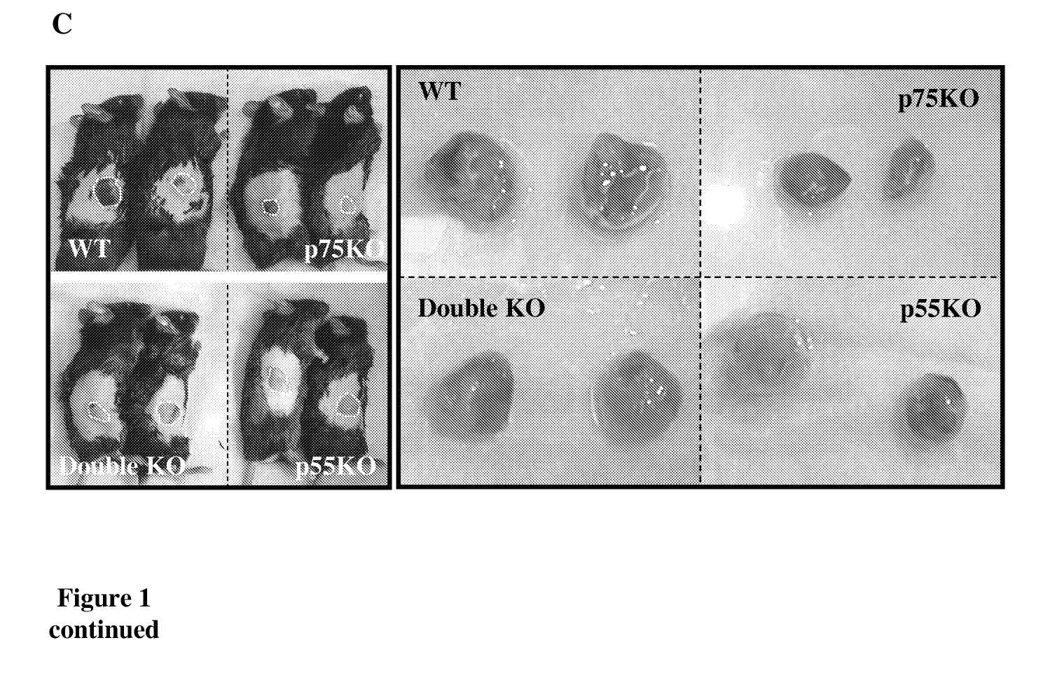

Tumors were larger in WT mice compared to those in p75KO and p55KO mice (FIG. 1A), suggesting that the host micro environment of p75KO and p55KO mice does not support tumor growth. Between days 0-7, tumors grew similarly in mice of all genotypes (FIG. 1B). However, tumors grew by ˜343% and ˜933% in volume in WT mice between days 14 and 21, respectively, in comparison to the tumors at day 7. In contrast, tumor growth was inhibited over the same time period in both p75KO mice (by 46% and 51% on days 14 and 21, respectively, p<0.05) and p55KO mice (by 42% and 38% on days 14 and 21, respectively, p<0.05), when compared to WT. In one of the three independent studies a group of TNFR double knockout mice were injected with LLCs. The kinetics of tumor growth and tumor volumes in the TNFR double knockout mice were similar to those in WT mice (FIG. 1C), suggesting that absence of signaling through one but not both TNF receptors is unsupportive of tumor growth. This analysis showed that, at least due to the altered microenvironment of the tumor tissue, tumor growth was inhibited at least two-fold in the absence of signaling by either TNFR1/p55 or TNFR2/p75 (FIGS. 1A and 1B), but not when both TNF receptors were concurrently inhibited (FIG. 1C).

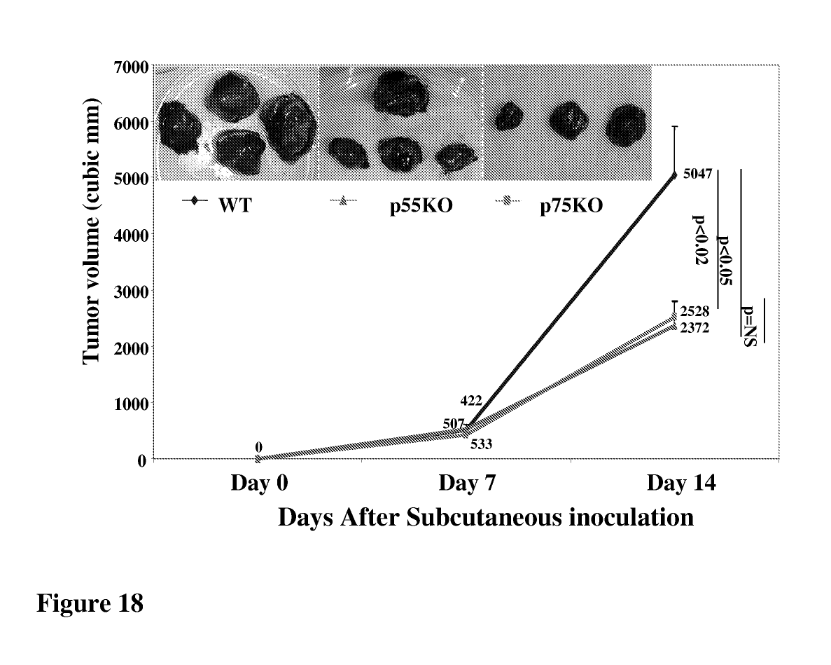

The inhibition of tumor growth over time by the host microenvironment (e.g. WT, p75KO or p55KO) was also demonstrated in WT, and TNFR1/p55 using mouse B16 cell melanoma line (B16), which is highly malignant and invasive (FIG. 18). For the experiment, at least 6-8 animals were used for each mouse genotype. Mice (6-8 weeks old) of all genotypes were injected with B16 melanoma cells (TNFR2/p75 mice, 2×10⁵ B16 cells were mixed with 50 micro liters of growth reduced Matrigel and injected into the flanks of WT, p75KO and p55KO mice). Before injection, the viability of B16 cells was determined to be more than 95% (trypan blue exclusion method). Tumor growth in mice was evaluated by measuring length, width and height of tumors weekly using electronic calipers and melanoma volumes were calculated using standard mathematical formulas for volume (i.e., L×W×H or V=4/3πr³; similar volumes were obtained by either method). Experiments were terminated on day 14 for two reasons: first, because tumor sizes exceeding 1 cm in diameter were present in WT mice, and, second, because two mice in the WT group died on day 14. The remaining WT mice showed signs of distress, (e.g., limited feeding and/or consumption). All mice were sacrificed according to the approved IACUC protocol. No mortality was observed in p75KO or p55KO mouse genotypes injected with B16 melanomas up to day 14 post-inoculation when the experiments were terminated.

Representative images of bisected tumors (FIG. 18; insets) of WT, p75KO and p55KO showed that on day 14 after inoculation of B16 melanoma cells the tumors were larger in WT mice as compared to p75KO and p55KO mice. The data show that between days 0-7 tumors grew similarly in mice of all genotypes. Tumors in WT mice increased to 1195% in size (12-fold) on day 14 compared to their size on day 7. In contrast, over the same time period, tumor growth was inhibited in both p75KO and p55KO mice by 53% on day 14 (p<0.02 and p<0.05 for p75KO and p55KO, respectively) compared to WT mice. Thus, between day 7 and day 14, melanoma volumes increased only 468% and 474% in p55KO and p75KO mice, respectively, compared to the 1195% increase in melanoma volume in WT mice. In this experiment all tumors were wild type and the tumor growth inhibition was dependent on the unsupportive microenvironment in p75KO and p55KO mice. These results indicate that in the absence of signaling via one of the TNF-α receptors (either TNFR1/p55 or TNFR2/p75) melanoma growth was inhibited more than 2.5-fold, suggesting that host microenvironment of p75KO and p55KO mice does not support melanoma growth.

### Example 2

### Decreased Vascularization and Increased Apoptosis was Observed in Tumors in p75KO and p55KO Mice

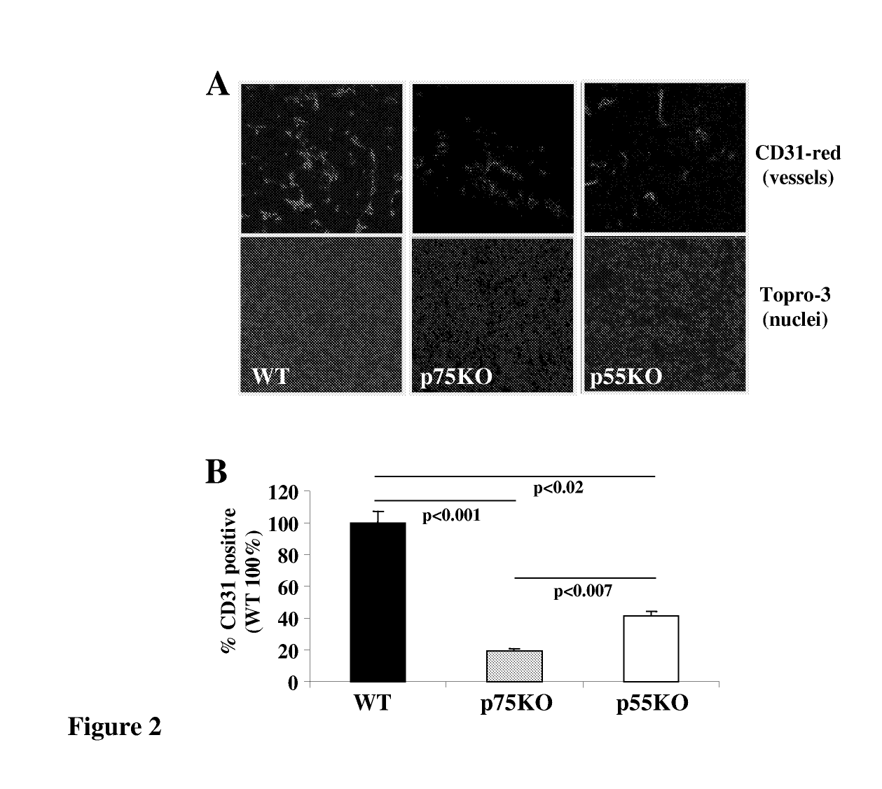

To understand the role played by angiogenesis in tumor growth and to determine the capillary density in different genotypes of mice, LLC tumors were bisected from WT, p75KO and p55KO mice, 14 days post-inoculation. Tumors from at least 5-6 mice from each group on day 14 post-inoculation were embedded in OCT compound and snap-frozen, then were fixed in methanol overnight and processed for immunohistology. Endothelial cells (EC) indicative of the capillary network were identified by immunostaining with CD31 antibody (Santa Cruz, Calif.), a well-accepted endothelial cell marker (FIG. 2A). Images obtained using laser scanning confocal microscopy (Zeiss, Axiovert 100, Thornwood, N.Y.) were evaluated in at least 5 animals/genotype using Image J program by measuring mean pixel intensity in 7-8 fields of 176,400 μm²/per mouse. Compared to tumors in WT mice, there were statistically significant 80% (p<0.001) and 60% (p<0.02) decreases in CD31 positive staining in tumors of p75KO and p55KO mice, respectively (FIG. 2B). There was an additional statistically significant decrease in CD31 positive staining in tumors of p75KO compared to p55KO mice (20% vs. 40%, respectively, p<0.007).

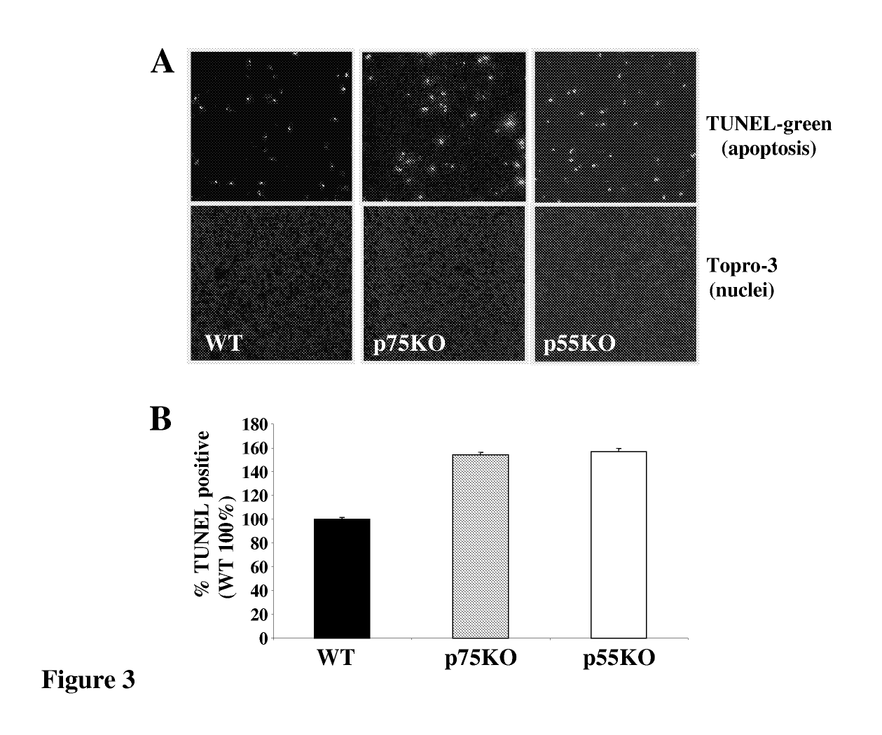

To determine the level of apoptotic processes in LLC tumors for all genotypes, bisected tumors from WT, p75KO and p55KO mice were processed for TUNEL staining. TUNEL positive (+) cells were identified by green nuclear staining (FIG. 3A). TUNEL (+) apoptotic cells were evaluated in at least 5 animals/genotype using Image J program by measuring mean pixel intensity in 7-8 fields of 176,400 μm²/per mouse. TUNEL-stained slides were evaluated using laser scanning confocal microscopy (Zeiss, Axiovert 100, Thornwood, N.Y.). Intensity of TUNEL staining in tumors of WT mice were set at 100% and TUNEL intensity in tumors of p75KO and p55KO mice were calculated as percent change from WT. Compared to tumors in WT mice, there was significant ˜50% increases in TUNEL positive cells in tumors of both p75KO and p55KO mice (FIG. 3B). The significant decrease in capillary density and vascularization of tumors together with an increased number of apoptotic cells in p75KO and p55KO mice accounts for the marked decrease in tumor volumes compared to WT.

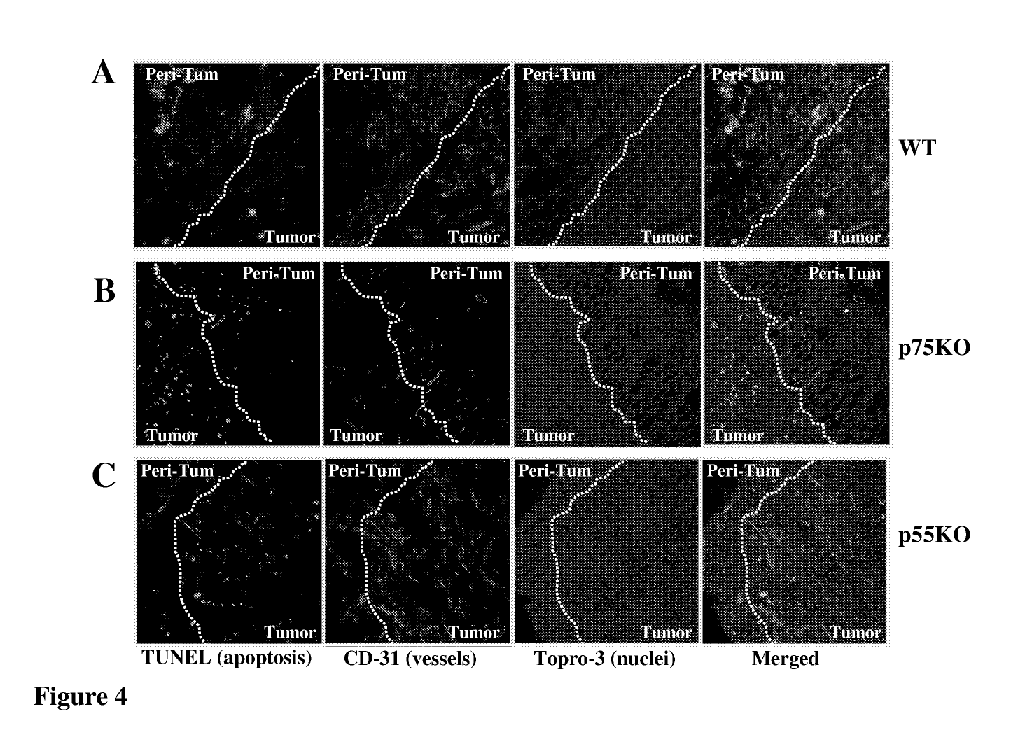

To evaluate the processes of apoptosis and tumor angiogenesis at the border-zone between tumor tissue and surrounding host tissue, peri-tumoral sections obtained from bisected tumors were triple-immunostained for TUNEL (green), CD-31 (red) and Topro-3 (nuclear staining-blue) (FIGS. 4A and 4B). TUNEL staining in tumors bisected from WT mice showed insignificant apoptosis in both peri-tumoral and tumor tissue, in contrast to an extensive capillary network in both areas, suggesting that host tissue supports tumor angiogenesis and that there is an insignificant amount of apoptosis in tumors of WT mice (FIG. 4A). p75KO mice showed a significant increase in apoptotic cells mainly in the tumor tissue, which was complemented by a significant decrease in capillary network in tumor tissue, and to lesser degree in peri-tumoral tissue of p75KO mice (FIG. 4B). p55KO mice showed similar results to p75KO tumors with respect to apoptotic cells in the tumor and peri-tumoral tissue and a decrease in capillary network in peri-tumoral tissue compared to tumor tissue (FIG. 4C).

There was little difference in capillary density in tumor tissue of p55KO compared to WT mice. Without wishing to be bound by theory, this result suggests that despite increased apoptosis in p55KO tumor tissue, tumor angiogenesis processes were not significantly affected, at least in the tumor tissue. Double positive cells (TUNEL/CD31-yellowish staining) indicated apoptosis of host p75KO EC's upon invasion of tumor tissue in p75KO mice (FIG. 4B, fourth column, tumor tissue area). This result suggests that there was a substantial deficiency in tumor angiogenesis in tumors of p75KO mice and an inability of host p75KO EC's to survive in the inhibitory tumor microenvironment.

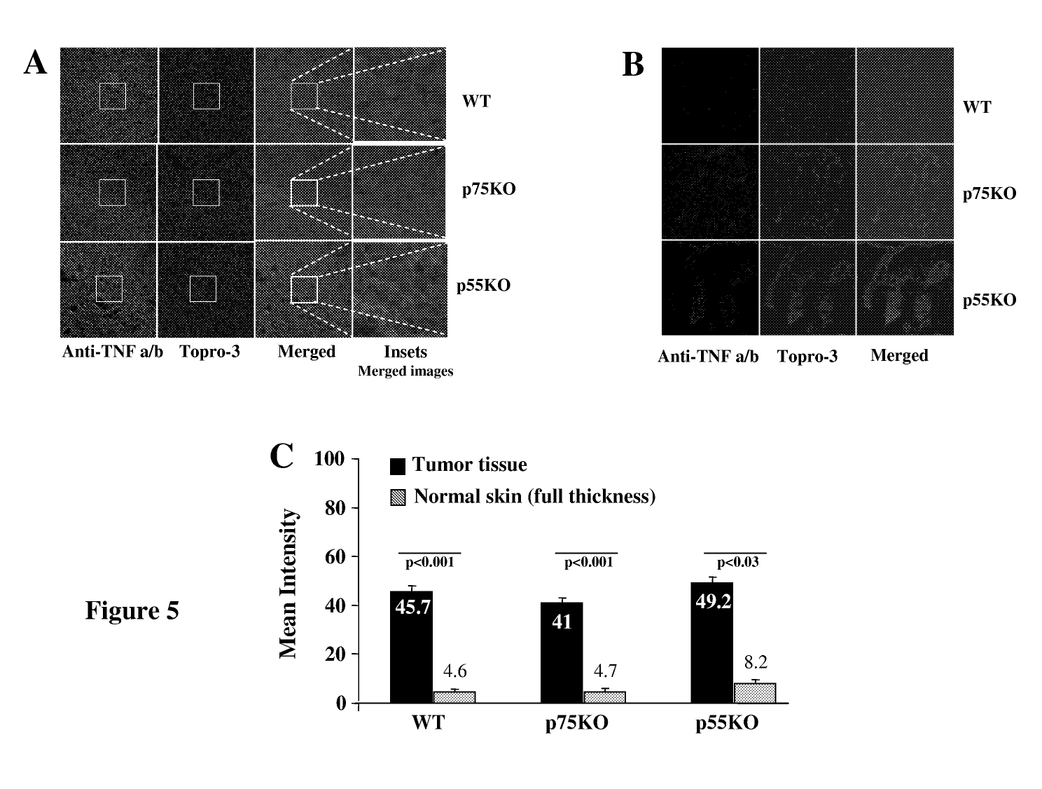

The levels of TNF-α (FIG. 5) in tumor tissue were also determined on the tumors bisected from different genotypes of mice. To determine TNF-α expression, bisected tumor tissue was processed for immunohistology using primary rat anti-TNF-α antibodies and species specific TRITC-labeled secondary antibodies. Representative images of TNF-αimmunostaining-red (left column), Topro-3 nuclear staining-blue (second column), merged images third column and higher magnification insets of merged images (far right column) are shown in FIG. 5A. Tumor tissue was immunostained for TNF-α antibodies and imaged using confocal microscopy (Zeiss, Axiovert 100, Thornwood, N.Y.). The level of TNF-αexpression in tumor tissues and normal skin samples were evaluated in at least 5 animals/genotype using Image J program by measuring mean pixel intensity in 7-8 fields of 176,400 μm²/per mouse. Normal skin harvested at least 3-4 cm away from the tumor area from the respective genotypes of mice was used as a control (FIG. 5B).

The intensity of TNF-α staining in tumor tissue from mice of WT, p75KO and p55KO was not statistically different among the three genotypes (FIG. 5C). Nor was the level of TNF-α immunostaining significantly different in normal skin samples from all three genotypes. TNF-α expression was 6-10-fold higher in tumor tissue from mice of all three genotypes compared to TNF-α expression in normal skin, and the increased expression of TNF-α in tumor tissue was statistically significant within genotype when expression in normal skin vs tumor tissue was compared in WT 10-fold (p<0.001), in p75KO 8.7-fold (p<0.001) and p55KO 6-fold (p<0.03).

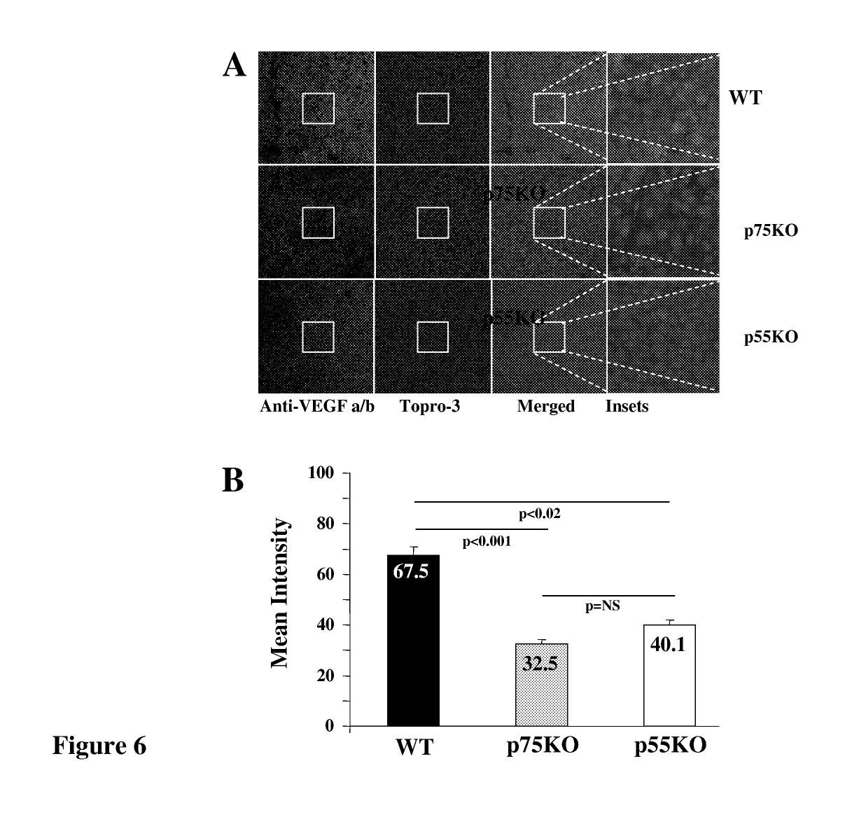

VEGF expression (FIGS. 6A and 6B) in bisected tumors was evaluated using specific VEGF antibodies. Bisected tumor tissue was processed for immunohistology using primary rat anti-VEGF antibodies and species specific TRITC-labeled secondary antibodies. All slides were evaluated using laser scanning confocal microscopy (Zeiss, Axiovert 100, Thornwood, N.Y.). VEGF expression levels in tumor tissues were evaluated in at least 5 animals/genotype using Image J program by measuring mean pixel intensity in 7-8 fields of 176,400 μm²/per mouse.

Compared to tumors bisected from WT mice the intensity of VEGF immunostaining decreased by more than 50% (p<0.001) in tumor tissue from p75KO; and by ˜40% (p<0.02) in tumor tissue from p55KO mice. There was no significant difference between VEGF levels in tumors of p75KO and p55KO mice. Without wishing to be bound by theory, these results suggest a deficiency in the expression of the angiogenic growth factor VEGF in the p75KO tumor tissue and to a lesser degree its expression in p55KO tumor tissue. This may, potentially, have a significant negative impact on the ability of the tumors in p75KO and p55KO mice to promote the development of pathological angiogenesis to support tumor growth and expansion. These histological studies support the decrease in tumor size observed in p75KO and p55KO mice (Example 1).

### Example 3

### p75KO Mice Had Fewer BM Derived Cells Incorporated into Tumors than WT Mice

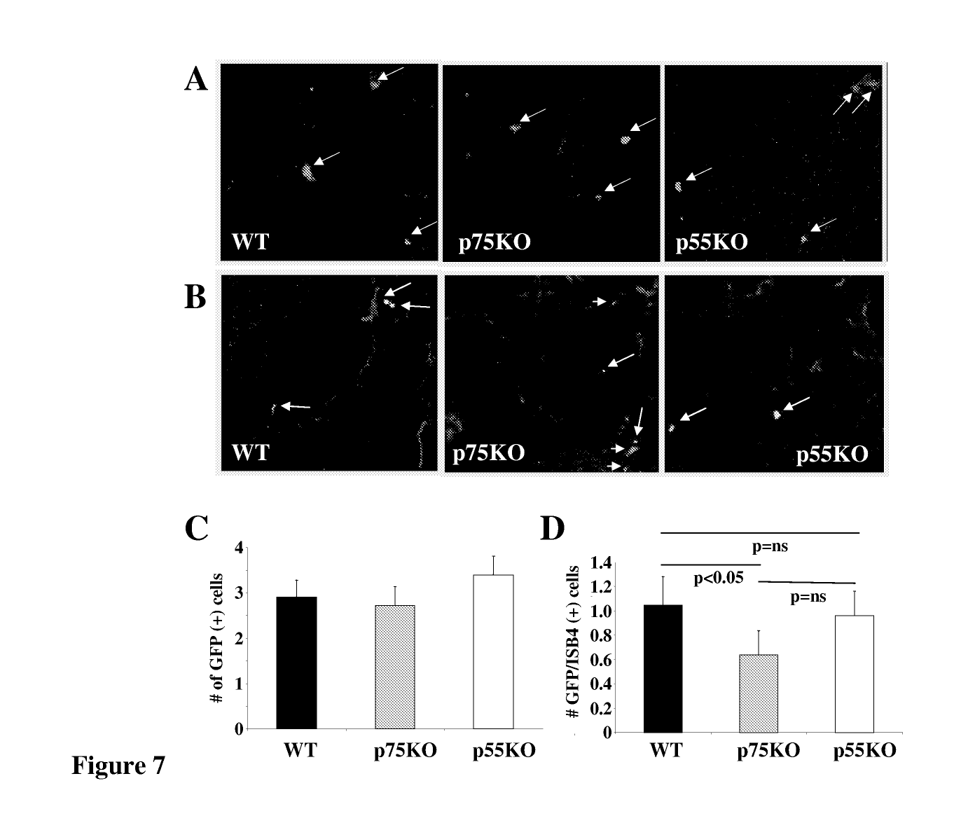

To track bone marrow (BM) derived cells into the tumor a BMT (bone marrow transplant) mouse model was used (FIG. 7A-7C). Briefly, WT, p75KO and p55KO mice were gamma-irradiated twice on day 1 with 9 Gr to kill their own bone marrow cells, and then transplanted with the mononuclear fraction of bone marrow cells from GFP positive mice of corresponding genotype. This procedure results in chimeric mice that have normal (non-GFP tissues and organs) but GFP bone marrow, which allows one to track the incorporation of BM-derived GFP positive cells into tumor tissue. To mark functional vessels, mice of all genotypes were perfused (5-10 min before sacrifice) with Rhodamine-labeled BS-1 lectin, a well-accepted endothelial cell marker. This perfusion method stains functional vessels, which are perfused by blood flow while the animals are still alive. LLC tumors bisected from WT, p75KO and p55KO mice were processed for immunostaining using Isolectin B4 staining. Tumors from all genotypes showed similar recruitment of GFP positive, BM derived cells (mostly EPC's) into the tumor (FIGS. 7A and 7C).

BM derived cells/EPC's were incorporated (in FIG. 7B, yellow cells, denoted by large arrows) and became a part of the functional vessels for both WT and p55KO genotypes. Compared to WT and p55KO mice there was a significant decrease (˜50%) in the incorporation of BM derived cells into the functional vessels network for p75KO genotype (FIGS. 7B and 7D). Without wishing to be bound by theory, this result suggests the functional deficiency of BM derived p75KO EPC in vivo.

### Example 4

### Analysis of Gene Expression in Tumor Tissue

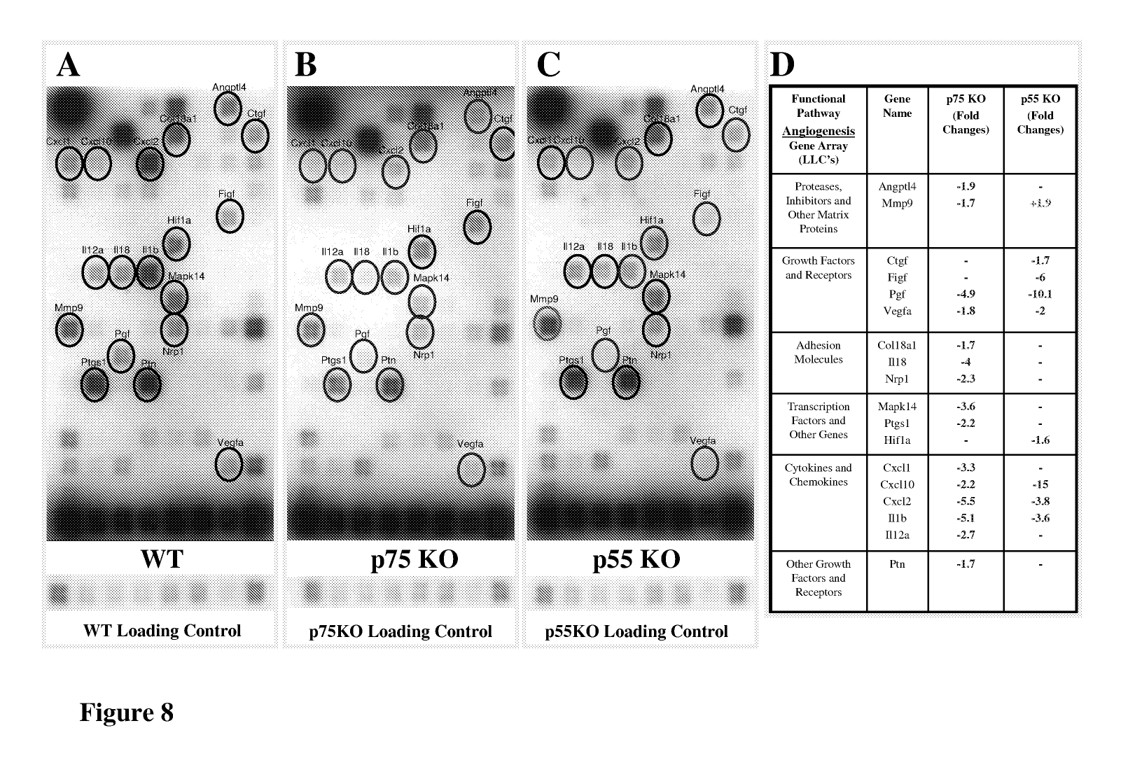

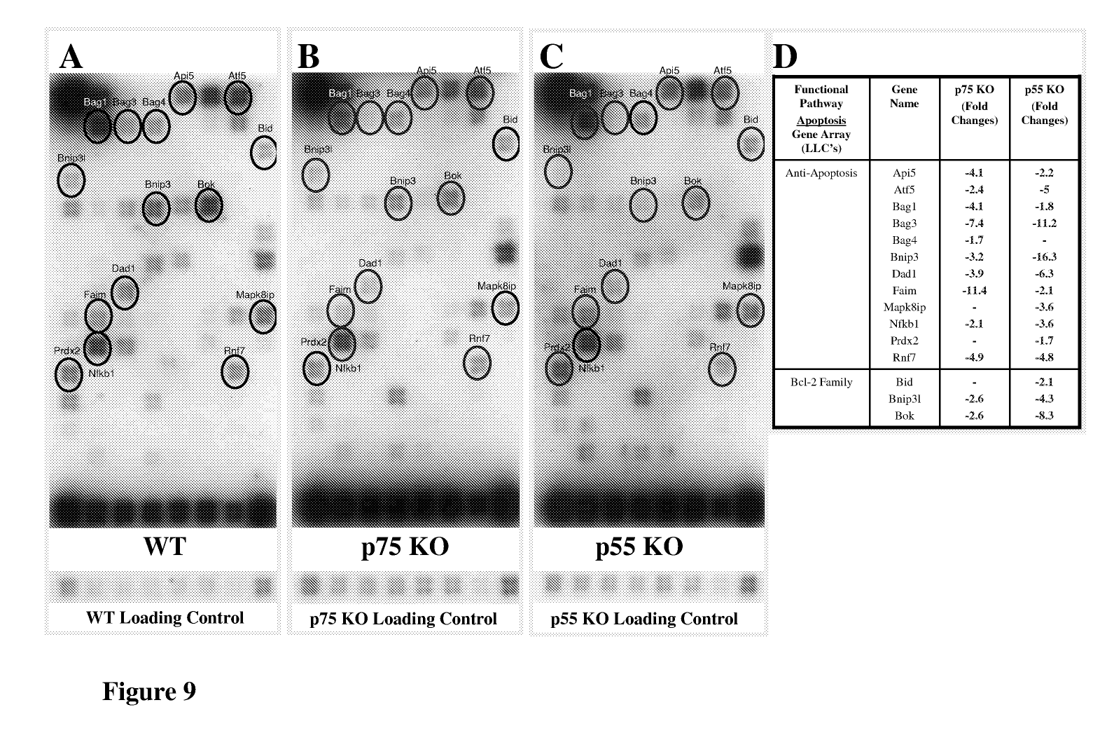

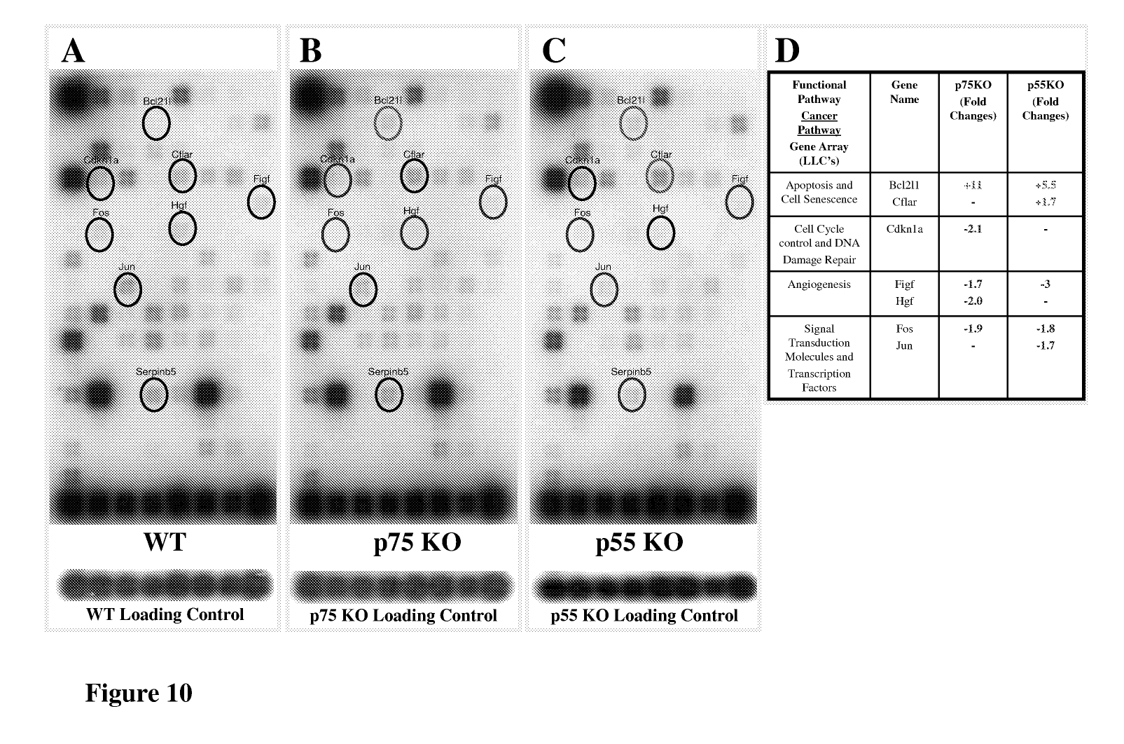

To understand the signaling pathways and genes involved during tumor growth, cRNA synthesized from isolated RNA was used for gene array analysis. Gene array analysis was carried out using commercially available Cancer Pathway Gene Expression Arrays, Mouse Angiogenesis Gene Arrays and Mouse Apoptosis Pathway Gene Expression Arrays. Tumors from all genotypes of mice studied (WT (C57BL/6), p55KO and p75KO) were dissected 14 days after inoculation. A pie-shaped piece from a tumor of each genotype was bisected and RNA was isolated. (FIGS. 8A-8D, 9A-9D, and 10A-10D). Total RNA was isolated from the tumors and converted to cRNA. The cRNA was amplified and labeled using the commercially available Superarray TrueLabeling-AMP 2.0 (Superarray, Catalog#GA-030). The cRNA sample was hybridized to the Cancer Pathway, Mouse Angiogenesis, and Mouse Apoptosis Gene expression arrays. Hybridization was carried out using the protocol provided in the commercially available Oligo GEArray Mouse Cancer Pathway Finder Microarray Kit from Superarray (Superarray, Catalog# EMM-033-4). Gene array expression on LLC and B16 tumors dissected from the three genotypes of mice was similarly performed.

The gene expression array films for both LLC and B16 tumors were analyzed using the Bio-Rad densitometry machine and software with different exposure times. The densitometric values of genes at positions 123 (Hsp90ab1), 124 (Hsp90ab1) and 127 (BAS2C) at the lowest exposure time were used to normalize the values of gene expression at higher exposure times between genotypes. The analysis provided the number of fold increase or decrease in gene expression of different genes between different genotypes for various pathways.

Analysis of the LLC tumors with Angiogenesis Pathway Gene Arrays indicated that the individual absence of either TNF receptor in the host tissue inhibited gene expression in several pathways that regulate tumor angiogenesis, as well as other cell survival processes such as proliferation, differentiation, inflammation, cell adhesion and regulation of gene transcription. Decreased gene expression was seen for angiogenenic growth factors, angiogenic cytokines and chemokine, adhesion molecule, matrix metalloproteinases and such potent angiogenic and survival transcription factors as Mapk14 (p38) and Hif-1alpha in tumors from the TNF knockout mice compared to those from the WT mice. The absence of TNFR2/p75 had a more significant inhibitory effect on several angiogenic pathways than the absence of TNFR1/p55 when both were compared to WT.

Analysis of the LLC tumors with Apoptosis Pathway Gene Arrays indicated that the individual absence of either TNF receptor in the host tissue significantly down regulated expression of anti-apoptotic and pro-survival genes. The individual absence of either TNF-αreceptor (p55 or p75) also down regulated expression of NFκB, a pro-survival and pro-angiogenic transcription factor that positively regulates expression of VEGF and diverse target genes that promote cell proliferation, regulate apoptosis, facilitate angiogenesis and stimulate invasion and metastasis.

Analysis of the LLC tumors with Cancer Pathway Gene Arrays indicated that the individual absence of either TNF receptor (p55 or p75) in the host tissue affected expression in several genes, when compared to LLC tumors in WT mice. In LLC tumors from p55KO mice Bcl211, Cflar, Figf, Fos, and Jun expression was altered, and in LLC tumors from p75KO mice, Bcl211, Cdknla, Figf, Hgf, and Fos gene expression was altered. Three of these genes, Bcl211, Figf, and Fos, showed similar changes in gene expression in tumors from both p55KO and p75KO mice. This result suggested that the different host environment did not affect specific cancer pathway(s) of LLC tumor cell line injected into mice of WT and TNFR1/p55 and TNFR2/p75 mice. The most significant change in gene expression observed between LLC tumors harvested from mice of different genotypes were increases in the expression of Bcl2-11, an apoptosis and cell senescence marker (11 and 5.5 fold, in p75KO and p55KO respectively).

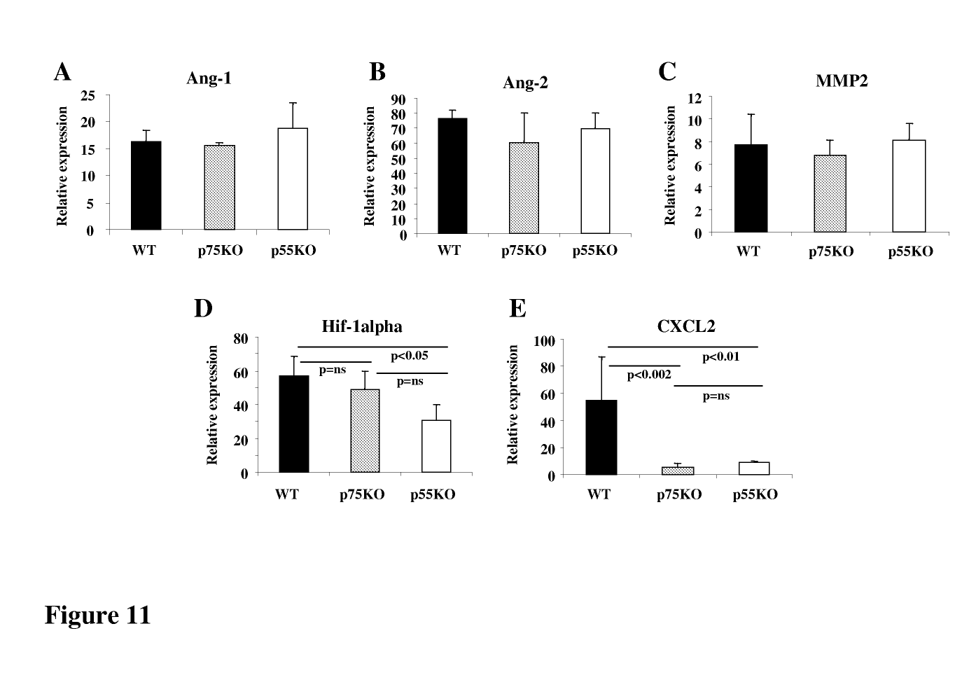

Quantitative RT-PCR results for certain genes of interest were performed to confirm the results of gene array analysis (FIG. 11A-11E). The cRNA generated from the LLC tumors of the different genotypes of mice; WT (C57BL/6), p55KO and p75KO, was used to perform reverse transcription reactions (TaqMan Reverse Transcription Reagents from Applied Biosystems). The cDNA obtained was analyzed using real time PCR. Reactions were carried out using commercially available SYBR Green real time PCR reaction for five selected genes: (three that did not show genotype-related changes in gene expression in the gene expression arrays—Ang-1, Ang-2, MMP2; and two that did show genotype-related changes in the gene expression arrays—Hif-1alpha, CXCL2). The reactions were set up using SYBR Green PCR Master Mix from Applied Biosystems, 10× Mouse genes QuantiTect Primer Assay from QIAGE and 20×18S Housekeeping Primer Mix from Applied Biosystems. A negative control containing no cDNA was used for each reaction. The samples were analyzed using Applied Biosystems 7300 Real Time PCR machine and the results were plotted as a graph between RE values (Relative Expression) and sample type (genotype of mice).

Quantitative RT-PCR (qRT-PCR) results showed that expression of Ang-1, Ang-2, and MMP2 did not change in the LLC tumor tissue between mice of WT, p75KO and p55KO genotype (FIGS. 11A-11C), confirming the results of gene array analysis (FIGS. 8A-8D, 9A-9D, 10A-10D). qRT-PCR showed that Hif-1alpha expression was significantly decreased in tumors from p55KO mice when compared with those from WT mice, whereas no significant difference was observed in the expression of Hif-1alpha gene in tumors obtained from p75KO when compared with those from WT (FIG. 11D), also confirming the results of gene array analysis (FIG. 8A-8D). CXCL2 gene expression was significantly decreased in tumor tissue from both p75KO and p55KO when compared with those from WT mice (FIG. 11E), again confirming the results of the gene array analysis (FIGS. 8A-8D).

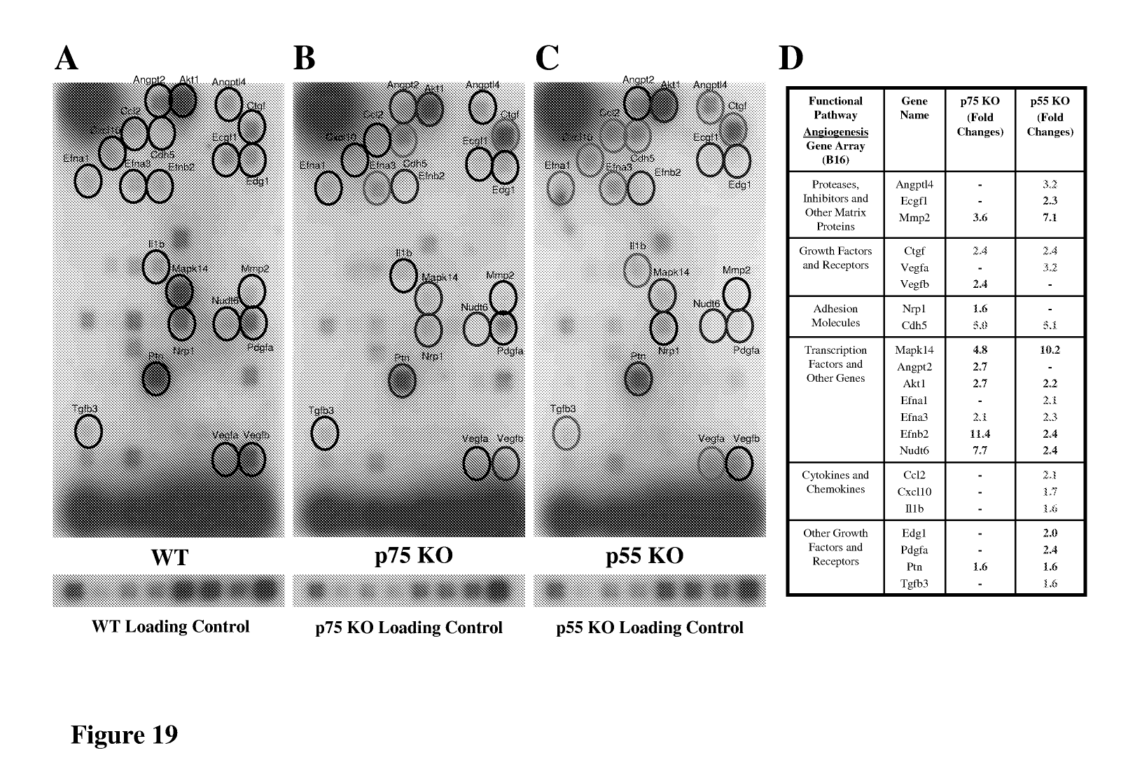

Analysis of the B16 tumors with Angiogenesis Pathway Gene Arrays indicated that the individual absence of either TNF receptor in the host tissue inhibited gene expression, albeit less than in LLC tumors, in several pathways that regulate tumor angiogenesis, as well as other cell survival processes such as proliferation, differentiation, inflammation, cell adhesion and regulation of gene transcription. Compared to tumors from WT mice, decreased gene expression was seen in tumors from the TNFR knockout mice for angiogenenic growth factors, adhesion molecules, matrix metalloproteinases and angiogenic and survival signal transduction molecules, such as Akt 1, and transcription factors, such as Mapk14 (p38) (FIG. 19A-19D). The absence of TNFR2/p75 had a more significant inhibitory effect on several angiogenic pathways than the absence of TNFR1/p55 when compared to WT. Many of the genes with changes in gene expression in the B16 tumors were distinctly different from the set of genes with altered gene expression in LLC tumors. This result indicated that LLC and B16 tumors exhibited their malignant phenotype through the regulation of different set of genes or pathways. However, regardless of the tumor type, the absence of signaling through TNFR/p75 or TNFR/p55 in the tumor microenvironment, is effective in decreasing the expression of genes involved in the regulation of tumor angiogenesis, thereby inhibiting tumor growth and propagation.

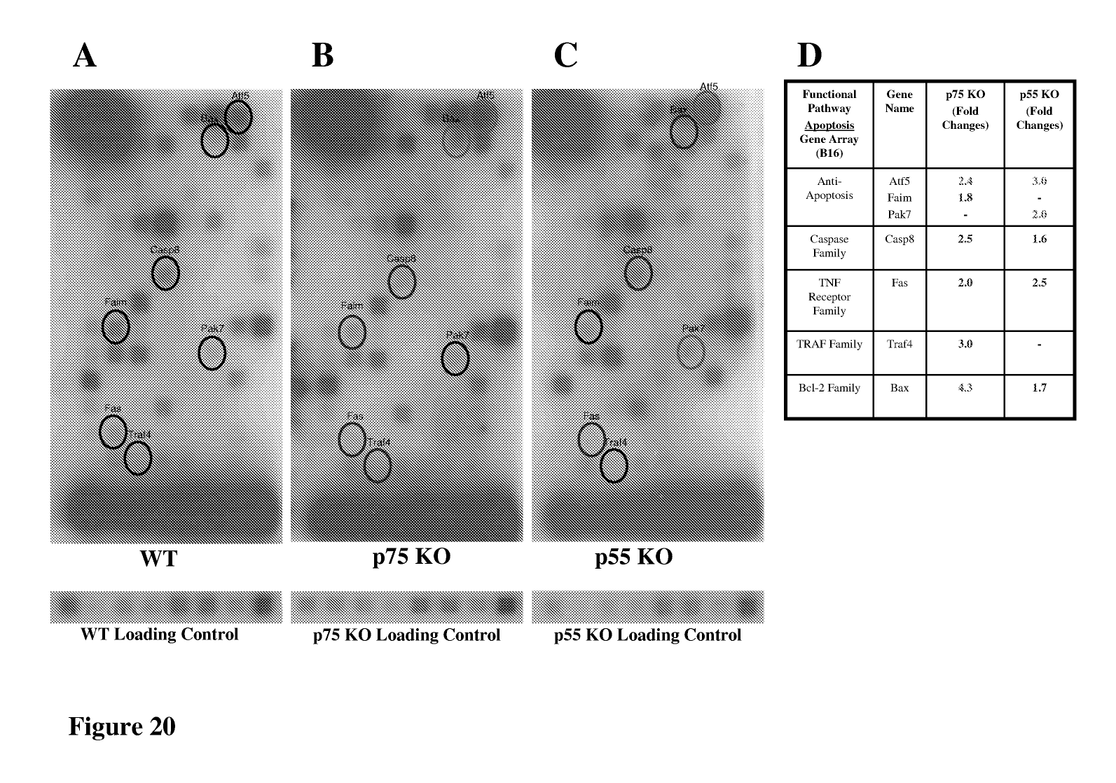

Analysis of the B16 tumors with Apoptosis Pathway Gene Arrays indicated that the individual absence of either TNF-α receptor in the host tissue did not alter the expression of anti-apoptotic and pro-survival genes (FIG. 20A-20D). At least in the set of 110 genes from the apoptotic pathways analyzed, only a few genes (about 5-6) showed altered gene expression. Because of the limited data, it was not possible to conclude the effect of the absence of either TNF-α receptor on the regulation of apoptosis pathways. Genome wide expression profiling could provide more information regarding the effect of the absence of either TNF-α receptor on the regulation of apoptosis pathways.

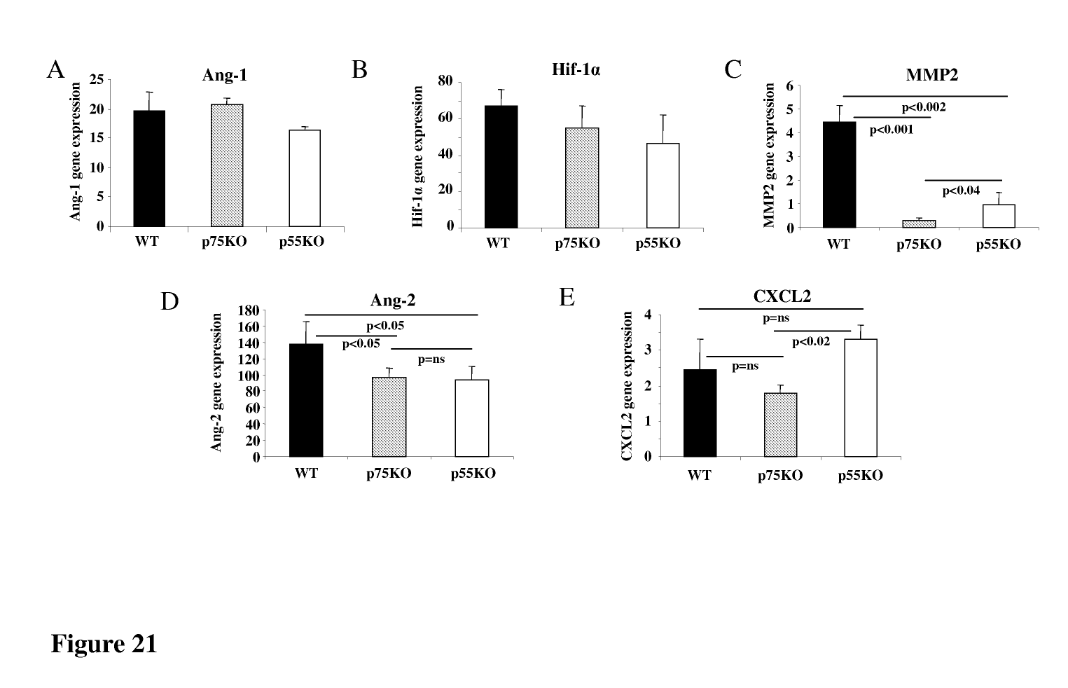

Quantitative RT-PCR for certain genes involved in tumor angiogenesis were performed, to further examine the results of the gene array analysis (FIG. 21A-21E). B16 tumors from WT (C57BL/6), p55KO and p75KO mice were dissected 14 days after inoculation. The cRNA generated from the LLC tumors of the different genotypes of mice; WT (C57BL/6), p55KO and p75KO, was used to perform reverse transcription reactions (TaqMan Reverse Transcription Reagents from Applied Biosystems). The cDNA obtained was analyzed using real time PCR. The cDNA obtained was analyzed using SYBR Green real time PCR reaction for five selected genes (two that did not show genotype related changes in gene expression—Ang-1, Hif-1α; and three that did show genotype-related changes—Ang-2, MMP2, CXCL2) to confirm gene array analysis results. The reactions were setup using SYBR Green PCR Master Mix from Applied Biosystems, 10× Mouse genes QuantiTect Primer Assay from QIAGEN and 20×18S Housekeeping Primer Mix from Applied Biosystems. A negative control containing no cDNA was used for each reaction. The samples were analyzed using Applied Biosystems 7300 Real Time PCR machine and the results were plotted as a graph between RE values (Relative Expression) and sample type (genotype of mice).

qRT-PCR results showed that expression of Ang-1 and Hif-1a did not change in the B16 tumor tissue between mice of WT, p75KO and p55KO genotype (FIG. 21A-21B). qRT-PCR results also showed that MMP2 expression was significantly decreased in B16 tumors from p75KO and p55KO mice when compared with tumors from WT mice (FIG. 21C). Finally, qRT-PCR results showed that CXCL2 gene expression was significantly increased in tumor tissue from both p55KO vs p75KO and WT mice (FIG. 21E). These results again confirm the results of the B16 gene array analysis (FIGS. 19A-19D) and that the absence of signaling through TNFR/p75 or TNFR/p55 in the tumor microenvironment is effective in decreasing the expression of genes involved in the regulation of tumor angiogenesis in B16 tumors.

### Example 5

### TNFR2/p75 Inhibition Reduced the Expression of Pro-Angiogenic and Pro-Survival Genes

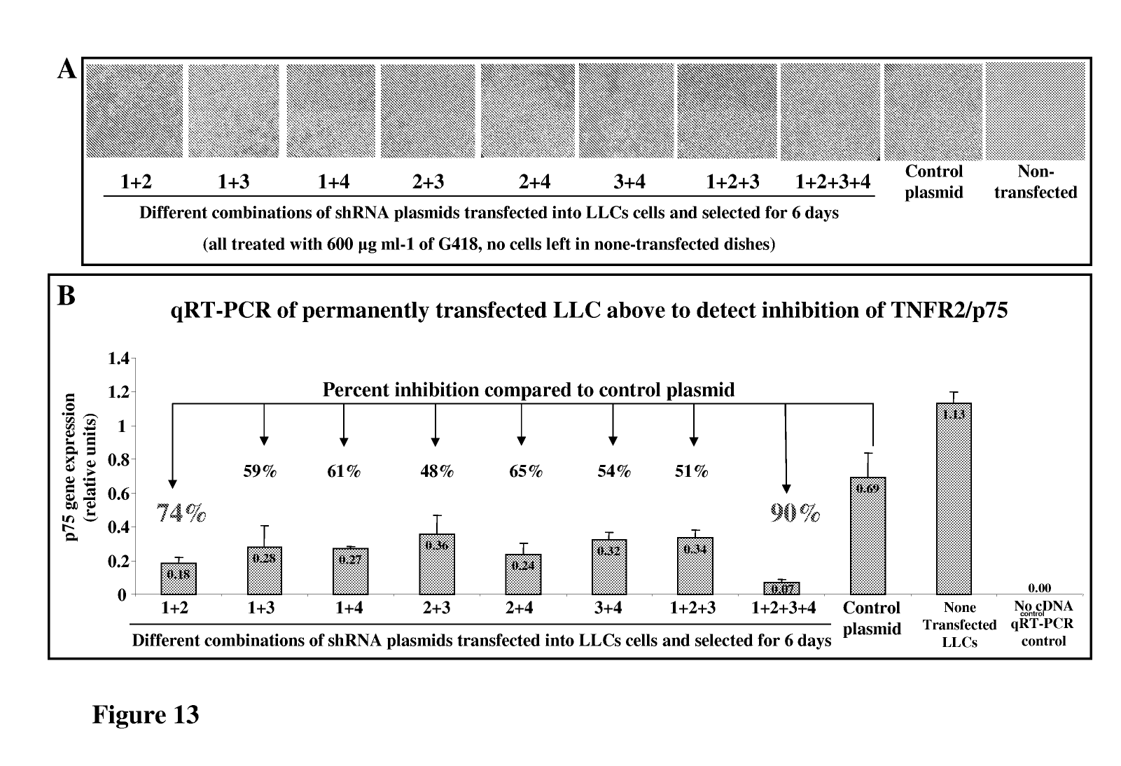

An in vitro study was performed to test the effect of inhibiting TNFR2/p75R expression. LLCs were transfected with p75 small hairpin RNA (shRNA) to knockdown TNFR2/p75 receptor expression. A dose response curve for genetecin (G418) was generated to determine the minimum concentration of G418 effective in the selection of LLC cells. The minimum concentration of G418 that kills all cells was found to be 600 μg ml⁻¹ for LLCs and 100 μg ml⁻¹ for endothelial cells. Transfections were performed according to a commercially available protocol using Effectene transfection agent (QIAGEN). The cells were grown for 2 days in 600 μg/mL G418 and then passed into 100 mm dishes at 40% confluence. The transfected cells were selected after six days in 600 μg/mL geneticin given on day 1 and day 3. Non-transfected cells were treated with the same concentration of G418 and were killed by day 6 (FIG. 13A; far-right panel). On day 6, stably transfected cells were harvested and processed for qRT-PCR for receptor analysis.

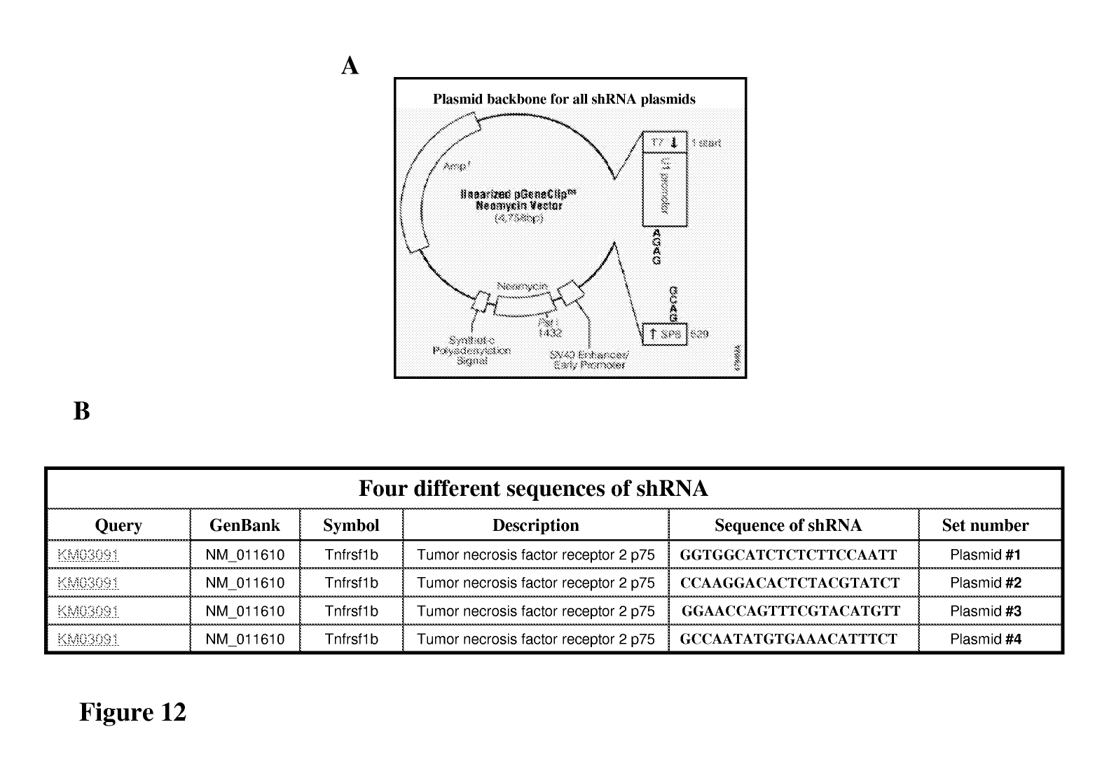

Transfection was performed using shRNA plasmids for TNFR2/p75 (Tnfrsflb) receptor (FIGS. 12A and 12B). LLC cells were plated and transfected with different combinations of shRNA plasmids (Table 1).

TABLE 1
shRNA encoded on plasmids.

| Plasmid | Query | Sequence of shRNA | Description | GenBank | Symbol |
|---|---|---|---|---|---|
| 1 | KM03091 | GGTGGCATCTCTCTTCCAATT | TNFR2/p75 | NM_011610 | Tnfrsf1b |
| | | (SEQ ID NO: 1) | | | |
| 2 | KM03091 | CCAAGGACACTCTACGTATCT | TNFR2/p75 | NM_011610 | Tnfrsf1b |
| | | (SEQ ID NO: 2) | | | |
| 3 | KM03091 | GGAACCAGTTTCGTACATGTT | TNFR2/p75 | NM_011610 | Tnfrsf1b |
| | | (SEQ ID NO: 3) | | | |
| 4 | KM03091 | GCCAATATGTGAAACATTTCT | TNFR2/p75 | NM_011610 | Tnfrsf1b |
| | | (SEQ ID NO: 4) | | | |

As a reference, LLCs were also transfected with a control plasmid containing a scrambled unrelated sequence GGAATCTCATTCGATGCATAC (SEQ ID NO: 5).

In total, nine different combinations of shRNA were tested for their inhibitory activity including 1+2, 1+3, 1+4, 2+3, 2+4, 3+4, 1+2+3, 1+2+3+4 and 5 (a control plasmid with scrambled, unrelated sequence). All combinations resulted in greater than 50% inhibition of constitutive levels of TNFR2/p75. A combination of shRNA plasmids 1+2 and 1+2+3+4 led to 74% inhibition and 90% inhibition of TNFR2/p75 expression, respectively as compared to the control plasmid (FIGS. 13A and 13B). In future studies LLCs and all other primary endothelial cells and human tumor cell lines were permanently transfected with these two combinations of TNFR2/p75 shRNAs, i.e., 1+2 and 1+2+3+4.

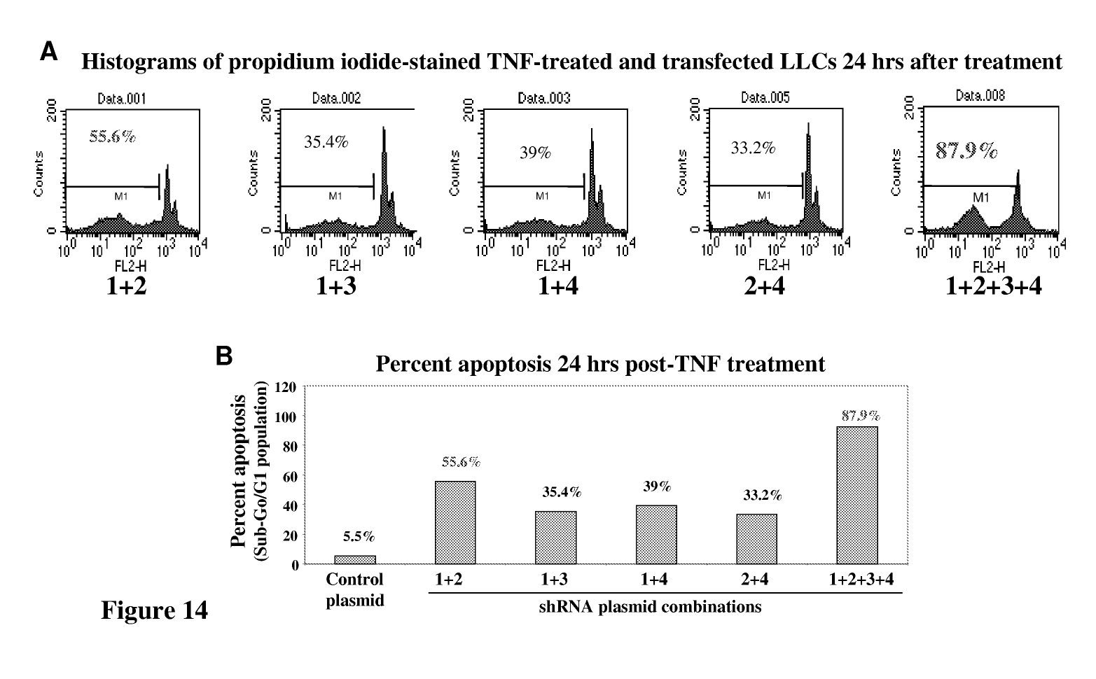

LLCs were treated for 24 hours with 1+2 and 1+2+3+4 combinations of TNFR2/p75 shRNAs for FACS analysis (FIGS. 14A and 14B). Different combinations of shRNA plasmids were transfected into LLC cells and selected for 6 days, and then treated with 80 ng/ml TNF (high dose) and harvested 24 hrs post-TNF treatment. FACS analysis was performed on the transfected LLC cells and analyzed using M1 gates for apoptotic cells (i.e., sub-G0/G1 population of cells with less than 2n DNA M1 gates representing, hence) of TNF treatment. TNF treatment for 24 hrs (80 ng/ml, high TNF dose) on stably transfected LLC revealed direct correlation with percent inhibition of TNFR2/p75 achieved by shRNA transfection (FIG. 14). In the transfected constructs used, the percent inhbition of the expression TNFR2/p75 (FIG. 13B) correlated with an increase in the percent apoptosis (FIG. 14A,B).

LLCs were treated for 24 hours with 1+2 and 1+2+3+4 combinations of TNFR2/p75 shRNAs for analysis of angiogenic gene expression by qRT-PCR. Two sets of LLCs transfected with TNFR2/p75 shRNA were prepared. One set of LLCs was treated with TNF-α (80 ng/mL) and the other was not. The LLCs used for angiogenic gene expression were harvested after 24 hours and 48 hours for qRT-PCR.

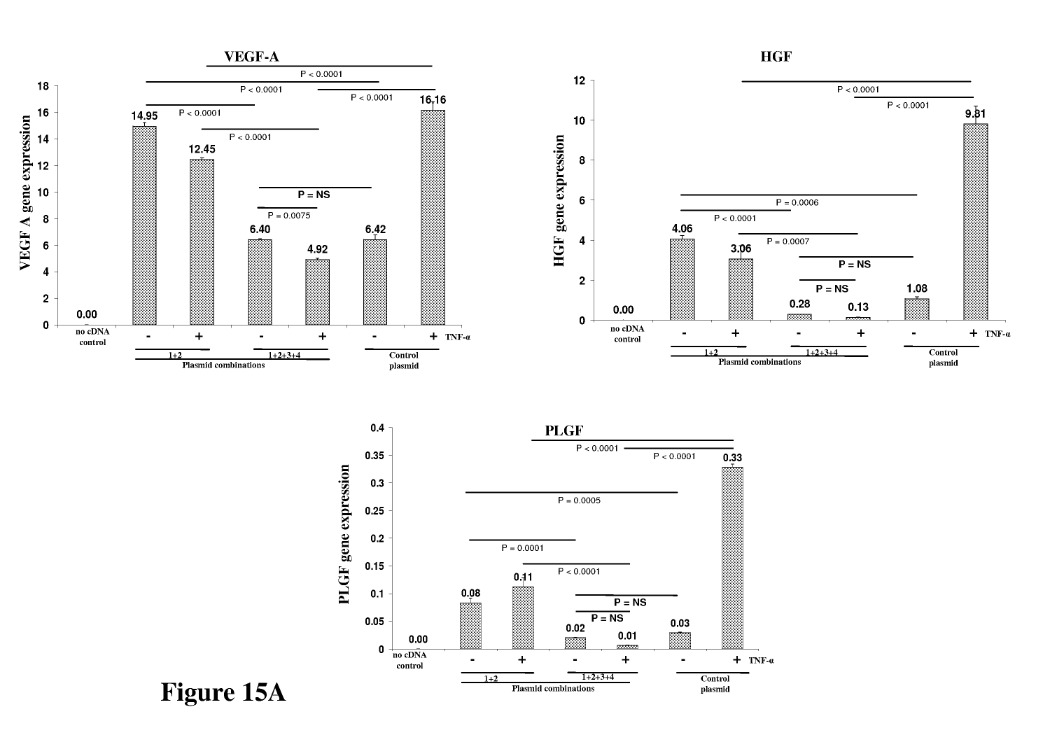

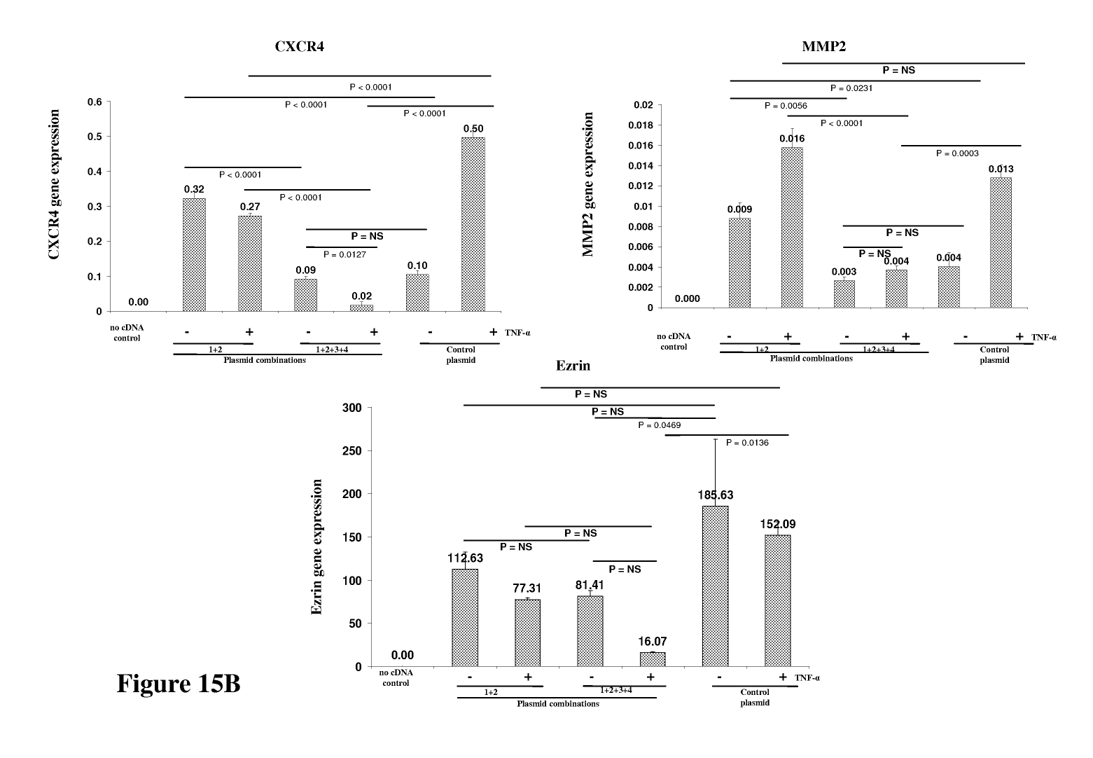

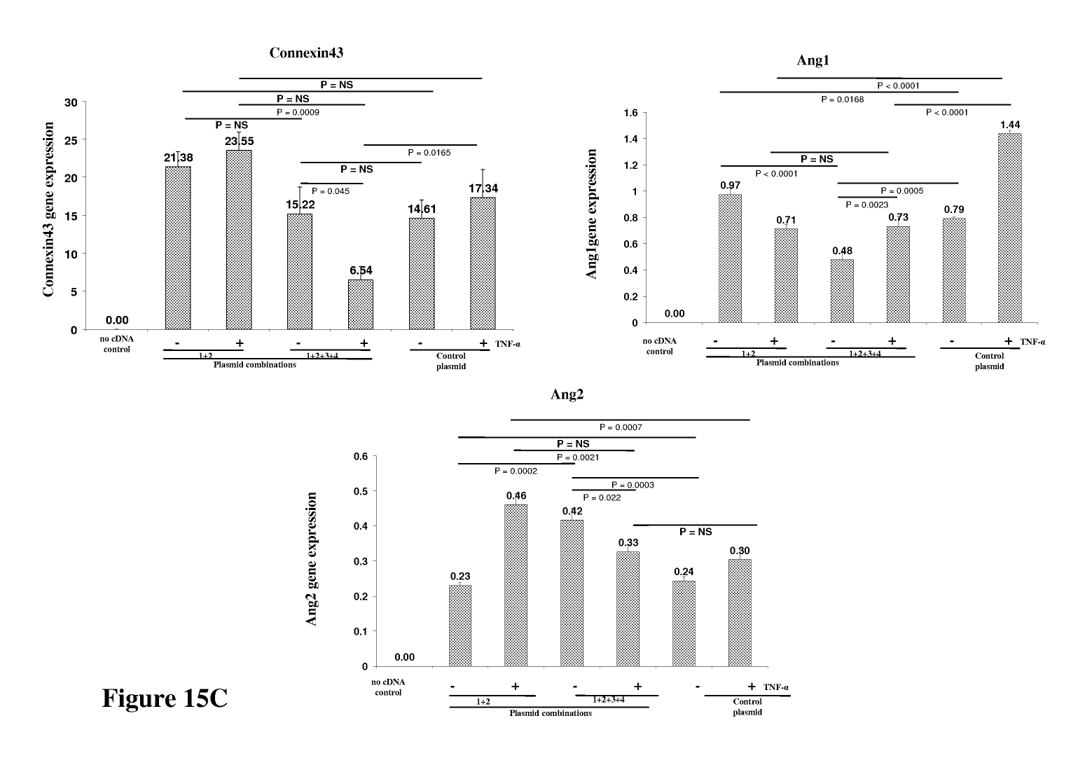

FIGS. 15A and 15B show a down regulation of various important genes involved in tumor growth and angiogenesis at 24 hrs after TNF treatment. LLCs that were transfected with TNFR2/p75 shRNA and then treated with TNF showed significant decreases in the expression of VEGF-1, HGF, PLGF, CXCR4, MMP2, Ezrin (FIGS. 15A and 15B) after TNF treatment. Decreases in the expression of VEGF-1, HGF, PLGF, CXCR4, MMP2, Ezrin were also observed in LLCs that were transfected with TNFR2/p75 shRNA but were not treated with TNF (FIGS. 15A and 15B). These data showed that inhibition of TNFR2/p75 expression in tumor cells inhibited the expression of several potent pro-angiogenic and pro-survival genes, which would result in reduced capillary network development and increased LLC apoptosis in vivo. Some of the genes (such as And-1 and Ang-2) were not affected by the inhibition of TNFR2/p75 signaling (FIG. 15C), suggesting no role of this receptor signaling in the regulation of these genes.

### Example 6

### EPCs from p75KO Mice Exhibit Loss of Endothelial Cell Functions

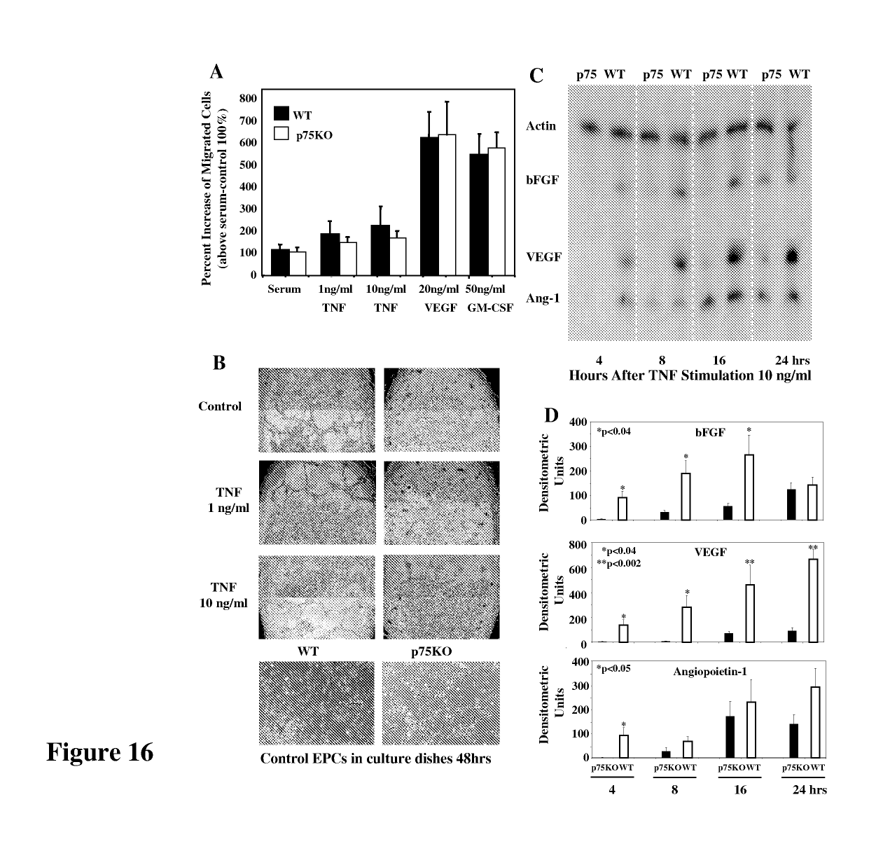

Chemotaxis and chemokinesis of WT and TNFR2/p75 EPCs in response to TNF-α (1 and 10 ng/ml), rmVEGF (20 ng/ml) and GM-CSF (50 ng/ml) was evaluated using a modified checkerboard assay using Coster Transwell chambers (6.5 mm diameter, 5 μm pore). Cells migrating into the lower chamber were collected in 50 μl of buffer and counted manually using hemocytometer and Coulter Counter. No difference was observed in the chemotactic activity between WT vs. p75KO EPCs in in vitro migration towards TNF, VEGF, and GM-CSF (FIG. 16A).

To examine the formation of tube-like structures, ex-vivo expanded WT and p75KO EPCs were seeded at 5×10⁴ cells/well on 4-well chamber slides coated with Matrigel (Collaborative Biomedical Products, Bedford, Mass.) and incubated for 12 hours in medium containing 5% FBS and supplemented with medium alone or 1 and/or 10 ng/ml of commercially available recombinant TNF-α (BD PharMingen). Cells in the chambers were examined and photographs were taken 12 hours post-stimulation. The cell's ability to form tube-like structures on VEGF-enriched matrigel is an important functional characteristic of ECs. EPCs from WT mice formed tube-like structures in control chambers and those treated with TNF, whereas EPCs from p75KO mice failed to form tube-like structures in either type of chamber, indicating a functional loss in the BM-derived EPCs of p75KO mice (FIG. 16B).

To examine mRNA expression of angiogenic factors in vitro in ex vivo expanded WT and p75KO EPCs treated with 10 ng/ml of recombinant TNF-α, Ribonuclease Protection Assay (RPA) was performed using a custom made angiogenic factor (VEGF, bFGF and Angiopoeitin 1) multiprobe DNA template (PharMingen). Total cellular RNA was isolated using Trizol reagent (Life Techonologies). [α ³²P]UTP (NEN) was used to synthesize in vitro transcribed antisense riboprobe and RPAs were carried out using RPA III TM kit (Ambion). Densitometric analysis revealed that TNF-induced expression of bFGF mRNA was abrogated in p75KO mice up to 16 hours after treatment and was only a third of the bFGF mRNA level of WT cells (FIG. 16C,D). The difference in VEGF expression in p75KO compared to WT mice was significant. VEGF expression was virtually undetectable in p75KO cells up to 8 hours after TNF treatment and it was still 8 fold lower by 24 hours in p75KO EPCs compared to WT EPCs (FIGS. 16C and 16D). TNF-induced mRNA levels of Angiopoeitin-1 were comparable at 8 and 16 hours in EPCs from p75KO cells compared to WT cells, and by 24 hours were reduced to half of the levels in WT cells (FIGS. 16C and 16D).

### Example 7

### NFκB Signaling in p75KO EPCs was Significantly Reduced

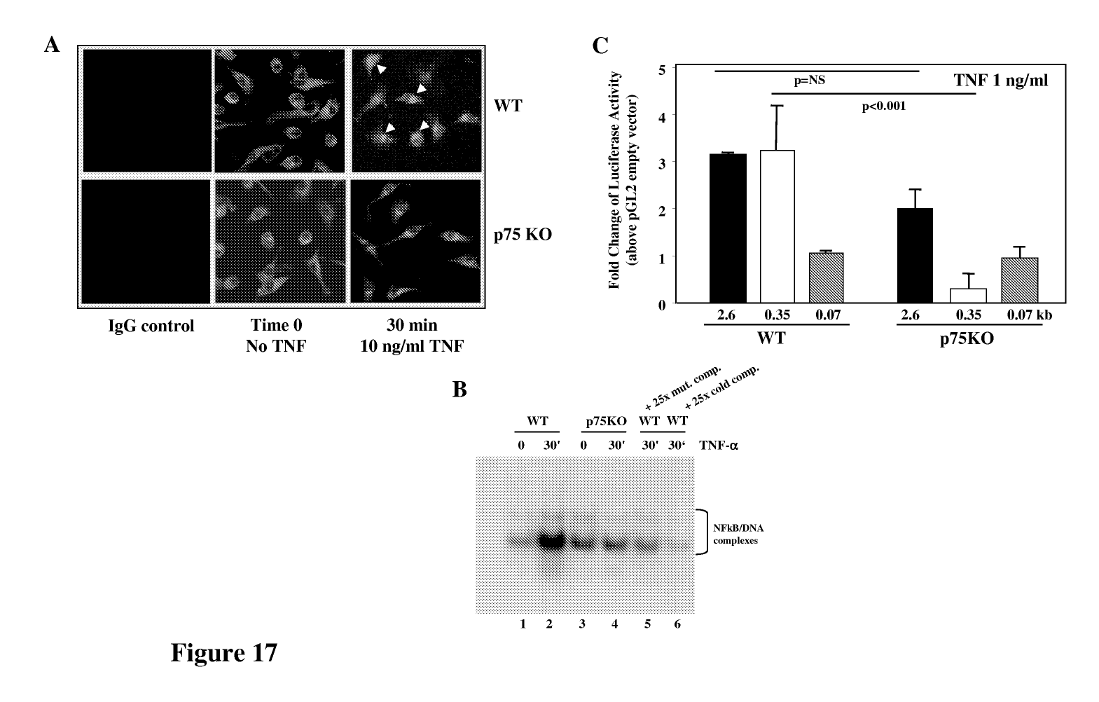

To examine the effect of TNF on NFκB nuclear translocation (NT) EPCs (grown in four well chamber slides) from WT vs. p75KO were treated with TNF (10 ng/ml) for 30 minutes and processed for immunostaining with NFκB p65 (Santa Cruz Biotechnology). Thirty minutes after TNF treatment NFκB NT was completely abrogated in EPCs from p75KO mice, whereas NFκB was translocated in the nucleus in 100% of EPCs from WT mice (FIG. 17A).

Electromobility shift assay (EMSA) was performed with NFκB consensus sequence to evaluate NFκB DNA binding activity. EMSA showed that TNF treatment failed to activate (and in fact decreased) NFκB DNA binding activities in p75KO EPCs at up to 30 min, whereas in WT cells TNF treatment increased (3 to 4-fold) the NFκB DNA binding activity in WT cells at 30 minutes (FIG. 17B). These results suggest that in the setting of TNF stimulation, NFκB signaling in p75KO EPCs is significantly reduced.

To examine the role of TNF on NFκB-mediated VEGF promoter activity, EPCs from WT and p75KO mice were transfected with a full length (2.6 kb) VEGF promoter-reporter construct (in basic pGL2 plasmid backbone), a deletion construct (0.35 kb) containing two putative NFκB sites and one Sp1 cluster, or an inactive deletion construct spanning up to −70 from transcription origination site and containing an incomplete Sp1 cluster (constructs described in Pa1 S, Datta K, Mukhopadhyay D. Central role of p53 on regulation of vascular permeability factor/vascular endothelial growth factor (VPF/VEGF) expression in mammary carcinoma. Cancer Res. 2001; 61:6952-6957). Twenty-four hours after transfection, cells were treated with TNF (1 ng/ml) and evaluated for VEGF promoter activity at 18 hours thereafter by measuring luciferase activity. TNF exposure resulted in a 2- to −3-fold increase in the activity of full-length (2.6 kb) VEGF promoter in p75KO EPCs compared with WT EPCs (FIG. 17C, black bars). In WT cells transfected with an NFκB construct (0.35 kb) there was a 3-fold increase in VEGF promoter activity compared with WT cells transfected with a full-length construct, suggesting that in WT cells under similar TNF treatment conditions, NFκB alone can activate the VEGF promoter to the same degree as full length promoter (FIG. 17C, compare black bars to gray bars in WT). In contrast, VEGF promoter activity was completely counteracted in p75KO cells transfected with NFκB construct, indicating that signaling through p75 receptor is required for TNF-induced activation of the VEGF promoter (FIG. 17C, compare black bars to gray bars in p75KO) and that NFκB may mediate the induction of VEGF expression through the TNFalpha/p75 pathway. No differences in VEGF promoter activity were observed between WT and P75KO EPCs transfected with inactive (0.07 kb) construct.

### Example 8

### Radiobiological Bystander Effects were Altered when TNF Signaling Via Either TNFR1/p55 or TNFR2/75 was modified

Experiments were performed to determine the radiobiological bystander effect of p75KO and p55KO cells. EPCs from WT, p75KO, and p55KO were treated with media from corresponding irradiated WT, p75KO, and p55KO EPC cultures.

EPCs from young (8-12 weeks) mice for WT, p75KO, and p55KO were obtained (crushed bone marrow), isolated, and plated into two 6-well dishes per mouse. Cells were grown on glass coverslips coated with 0.2% gelatin (one coverslip per well). Of the two E-well dishes one served as the un-irradiated cells on to which irradiated/conditioned media transfer would be performed and the other dish was gamma irradiated at 1Gy for all genotypes. Post seeding cells were cultured for 5 days before initiating the study to attain confluence.

For the bystander effect study, medium transfer experiments were performed. On the day of the study (5 days after initial plating), media in all wells was changed with fresh media (3 ml) including control (CTRL) wells. After change of media, cells were incubated for 1 hr prior to irradiation. For control (CTRL) wells, the coverslip was transferred into a 35 mm dish with fresh media. The rest of the cells in the 6-well dishes were irradiated at 1Gy. At respective time points post irradiation (CTRL-medium from none irradiated cells, 5 hr, Day 1, Day 3 and Day 5 —medium collected from irradiated cells), filtered media (0.22 μm filter) was transfer onto non-irradiated EPCs (˜2 ml of irradiated/conditioned media). Non-irradiated cells were incubated for 24 hrs in conditioned media before collecting the coverslips for staining for presence/decay pH2AX foci (pH2AX foci correlate well with repair of DNA double strand breaks and are indirect indicator of DSB repair/decay). Coverslips at each time point were fixed and stained for pH2AX+Topro-3 (nuclear staining). Results of pH2AX foci were confirmed by co-localization of the foci using another marker for DSB-p53BP1. 100× images obtained for all samples were analyzed using computer assisted image analysis for foci count. Statistical analysis was performed using ANOVA/ANCOVA Fisher's PLSD (StatView statistical package). Statistical significance was assigned when p<0.05.

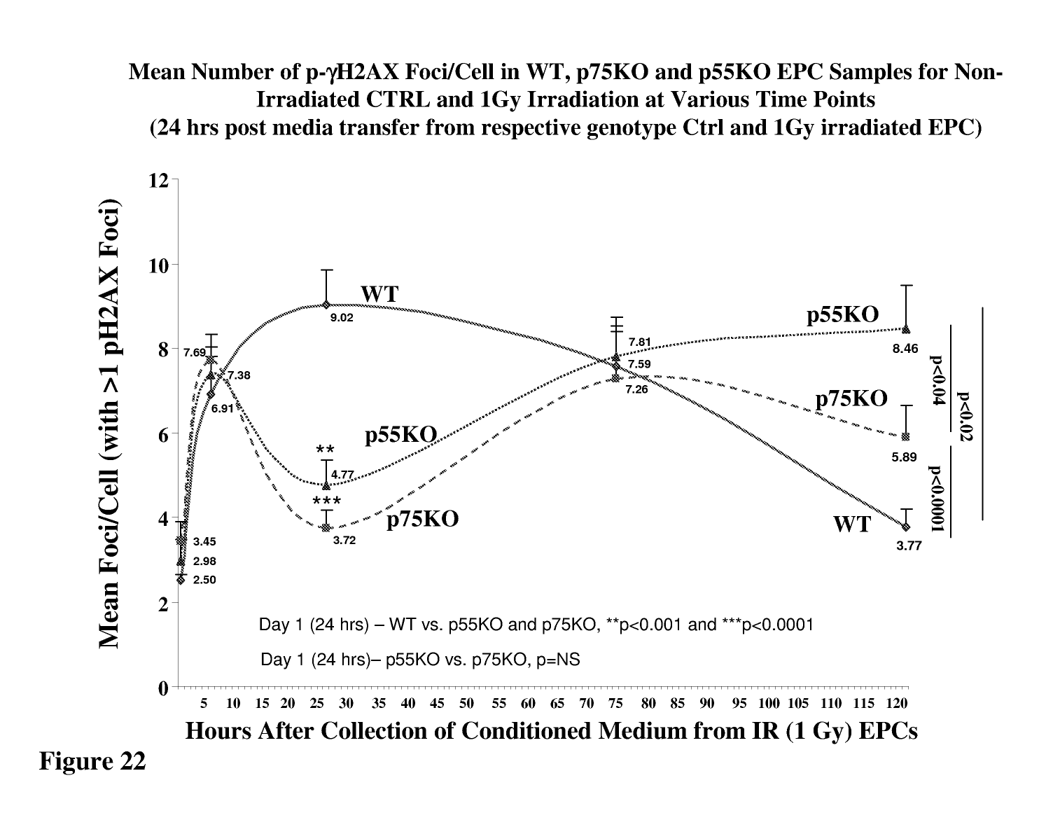

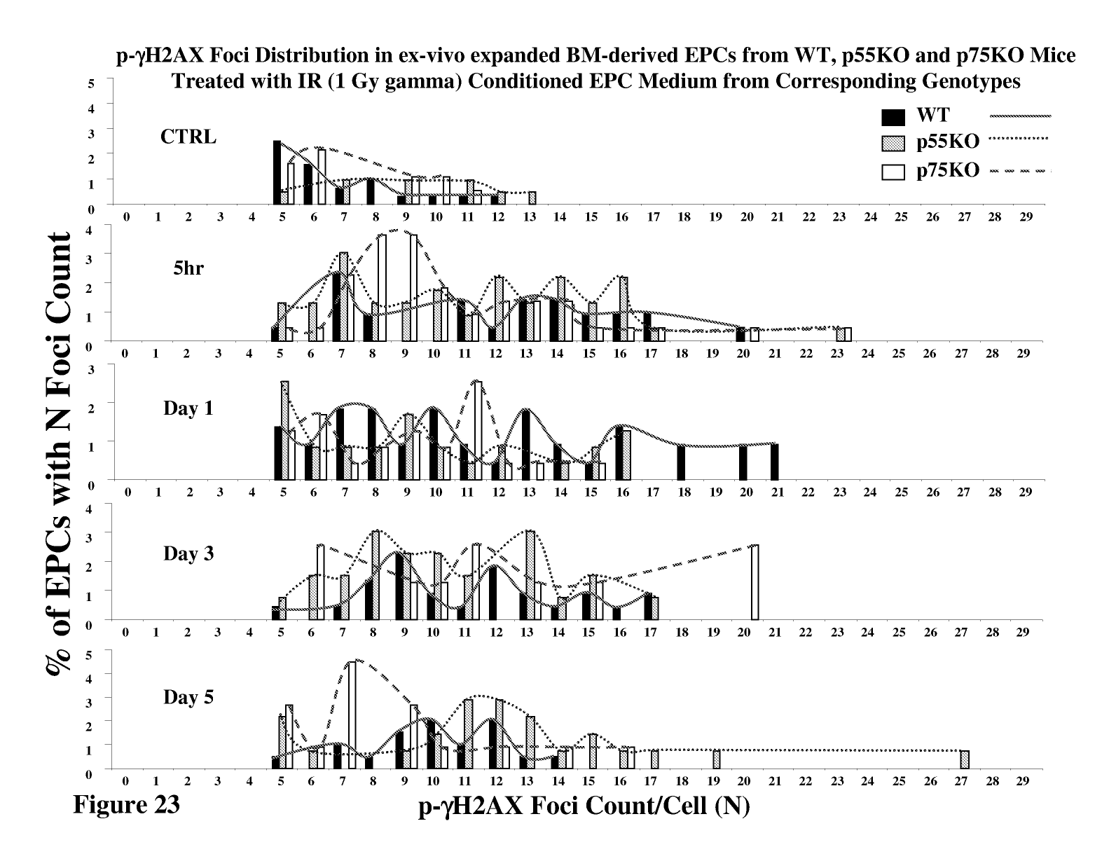

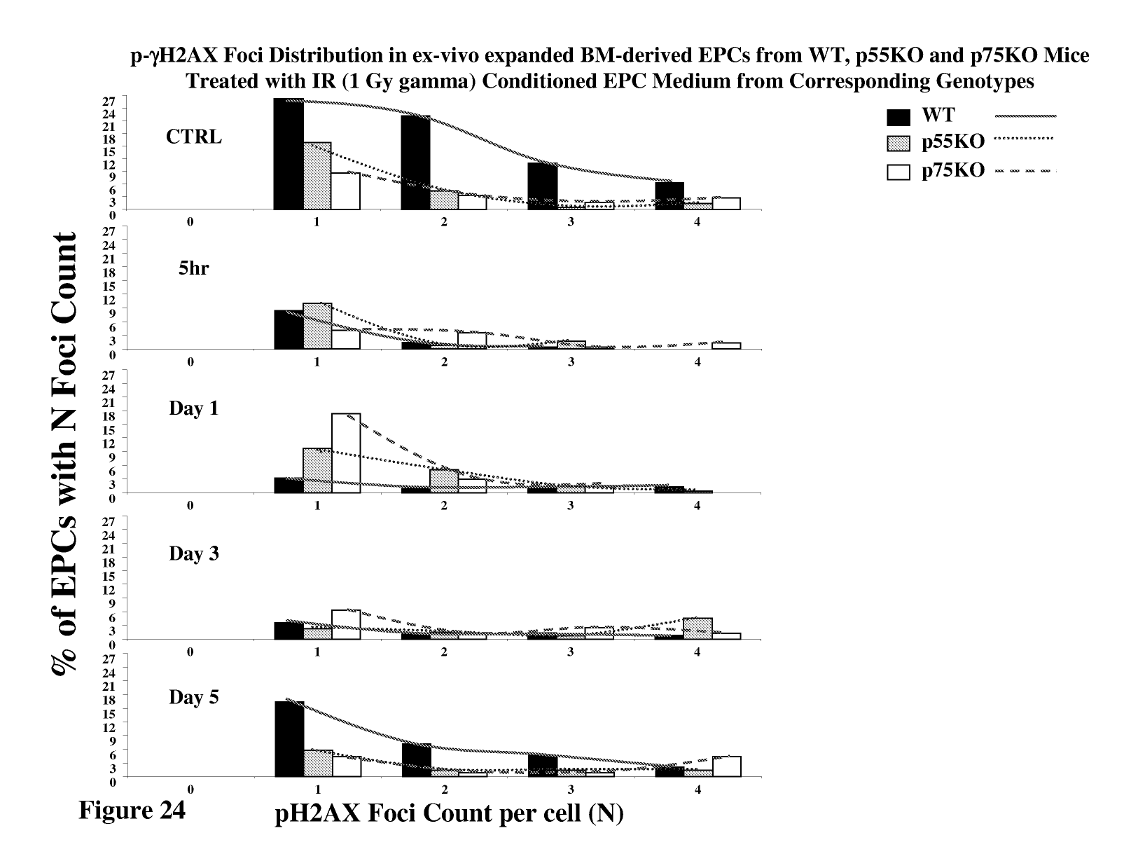

In the absence of either of TNF receptors (p55 or p75) there was a significant decrease, compared to WT, in the formation of p-γH2AX foci between 5-24 hrs after adding IR-conditioned medium to naïve BM-derived EPCs. This result indicated that TNF and signaling via either of TNF receptors was necessary for development of radiobiological bystander responses in non-irradiated BM-derived EPC (FIGS. 22 and 23). However, in the absence of either of TNF receptors (p55 or p75) there was a significant increase, compared to WT, in the formation of p-γH2AX foci between 1-5 days after adding IR-conditioned medium to naïve BM-derived EPCs. This result indicated that TNF and signaling via either of TNF receptors was delayed (FIGS. 22 and 23). Thus, bystander responses in non-irradiated BM-derived EPCs were amplified. Without being bound to a particular theory, a continuous increase in the number (N) of p-γH2AX foci/Cell between 1-5 days in naïve p55KO BM-derived EPCs is indicative that unopposed (by p55, mainly apoptotic) signaling via p75 (that is mainly survival signaling) in p55KO EPCs played an important role in delayed bystander responses.

Modification of TNF signaling via TNFR1/p55 or TNFR2/75 modulated radiobiological bystander effects. Understanding the roles of TNFR1/p55 or TNFR2/75 can be used to prevent delayed bystander effect-induced damage to naïve BM-derived EPC in normal tissue and to increase DSB, and, thus, to increase apoptosis in cancer cells.

### Example 9

### Tumor Growth was Inhibited in p75KO Mice and p55KO Mice

Experiments were performed to determine the effect of p75 knockdown (KD) in LLC tumors in both WT and p75KO host microenvironments. Additionally, experiments were performed to determine the effect of TNF injection into tumors (WT and p75 KD LLC's) in WT and p75KO host microenvironments.

Cells (5×10⁵) of un-transfected LLC's (WT; control) and p75 receptor knockdown (KD) LLC's using the shRNA plasmid combination (1+2, ˜60-70% inhibition of p75 receptor in LLCs) were mixed with 1000 of growth reduced Matrigel and injected subcutaneously into the right flanks of WT and p75KO mice. The mouse models were used to compare the combined effect of host microenvironment (e.g., WT or p75KO) and p75 receptor KD cells on the growth and development of tumors over time. At the time of injection, mice of both genotypes were 10-12 weeks old. Before injection, LLCs were tested for viability by trypan blue exclusion method. It was found that in all injection samples the viability of cells was more than 95%. Tumor growth was evaluated by measuring length, width and height of tumors at Day 0, 6, 8, 11 and 14 using electronic calipers. Tumor volumes were calculated using mathematical formula (V=0.52×L×W²). Mortality was monitored in all groups for the entire duration of the study. Post day 14, the tumors were allowed to grow for another 10 days prior to mouse recombinant (mr) TNF treatment (intra-tumoral injection) on Day 24. mrTNF was administered 4 times intra-tumoral from day 24 through 31, based on body weight calculation of 12.5 μg/kg/per injection. This is a dose of mrTNF that is twice as low as published intraperitoneal (IP) or intravenous (IV) injections in mice (Creasy A A., et al., 1986). Total intra-tumoral mrTNF dose administered over 7 days was 1 μg/mouse.

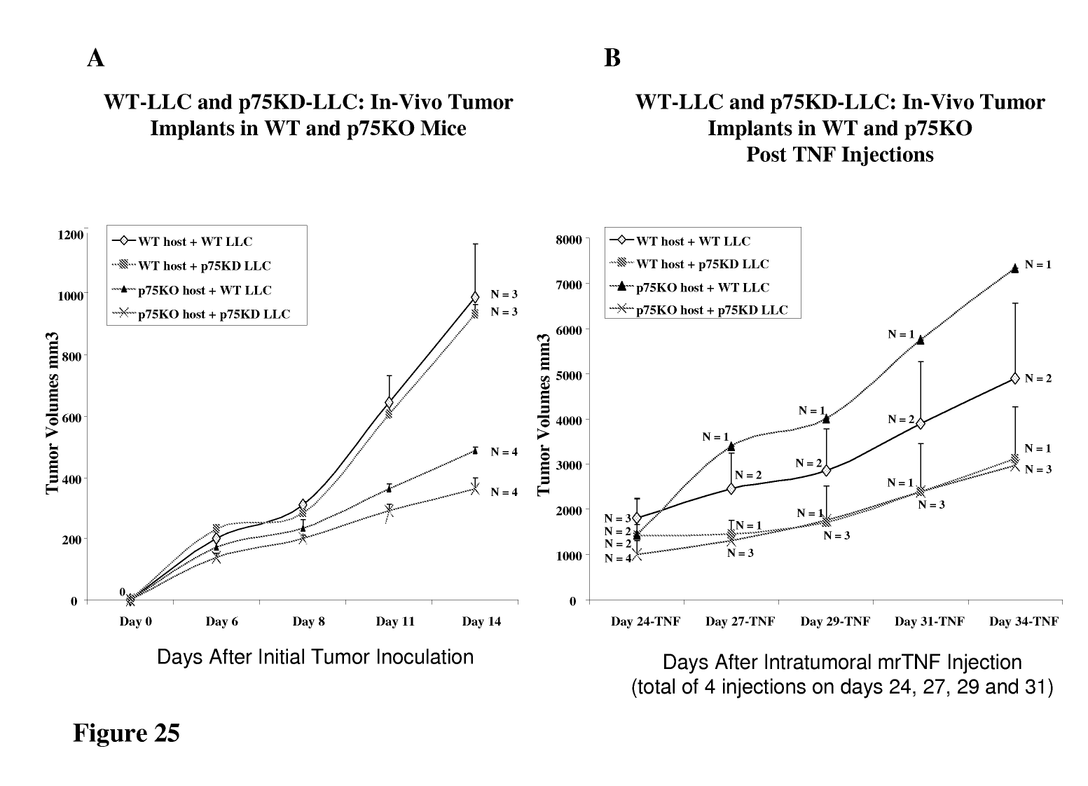

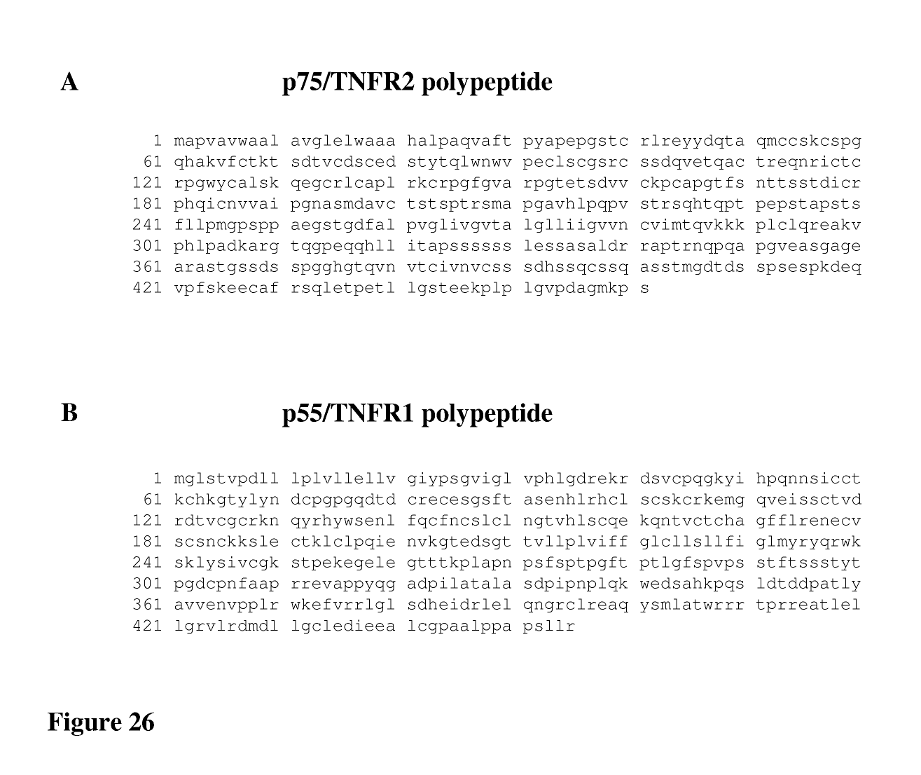

As shown before, p75 knockout host microenvironment reduced growth (˜60%) of unaltered LLCs (FIG. 25A). However, there was an additional decrease in tumor growth (˜70%) when the microenvironment was p75KO and LLCs were p75 receptor knockdown (KD) (FIG. 25A). Intratumoral low dose TNF injection increased survival (75%) of mice and inhibited tumor growth significantly in p75 KD LLC tumors (WT host+p75 KD LLC; p75KO host+p75 KD LLC) (FIG. 25B). When p75 KD LLC tumors were treated with low dose TNF, the reduction in tumor size was comparable in both WT and p75KO host microenvironments.

Thus, the results show that inhibiting TNFR2/p75 expression in tumor tissue in vivo is effective for treating solid cancer treatment. Additionally, inhibiting TNFR2/p75 expression in tumor tissue in vivo in combination with intratumoral administration of low non-toxic human recombinant TNF is a novel therapeutic modality for solid cancer treatment.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method of reducing neoplastic cell survival or proliferation, the method comprising contacting a neoplastic cell with an agent that selectively reduces the expression or activity of a p75 or p55 TNF-α receptor in the neoplastic cell relative to an untreated control cell, thereby reducing neoplastic cell survival or proliferation.

2. The method of claim 1, wherein the method selectively reduces the expression or activity of the p75 TNF-α receptor in the neoplastic cell while the expression or activity of the p55 TNF-α receptor is not disrupted.

3. The method of claim 1, wherein the method selectively reduces the expression or activity of the p55 TNF-α receptor in the neoplastic cell while the expression or activity of the p75 TNF-α receptor is not disrupted.

4. (canceled)

5. The method of claim 1, wherein the agent is an inhibitory nucleic acid molecule selected from the group consisting of an antisense molecule, an siRNA, and an shRNA that is complementary to at least a portion of a p75 or p55 TNF-α receptor nucleic acid molecule.

6. (canceled)

7. The method of claim 5, wherein the inhibitory nucleic acid molecule comprises or consists essentially of a nucleic acid molecule with a sequence selected from the group consisting SEQ ID NO: 1, SEQ ID NO: 2, SEQ ID NO: 3, and SEQ ID NO: 4.

8. The method of claim 1, wherein the agent is an antibody or fragment thereof that selectively binds to the p75 or p55 TNF-α receptor.

9. (canceled)

10. The method of claim 1, wherein the method increases cell death or reduces blood vessel formation in a neoplasia.

11. A method of inhibiting angiogenesis or increasing cell death in a neoplasia, the method comprising contacting a neoplastic or endothelial cell with an agent that selectively reduces the expression or activity of a p55 or p75 TNF-α receptor in the neoplastic or endothelial cell relative to a reference, thereby inhibiting angiogenesis or increasing cell death in the neoplasia.

12-30. (canceled)

31. A method of treating a subject with a neoplasia, the method comprising administering to the subject an effective dose of an agent that selectively reduces the expression or activity of a p55 or p75 TNF-α receptor in a cell, thereby treating the subject with the neoplasia.

32-34. (canceled)

35. The method of claim 31, wherein the agent is an inhibitory nucleic acid molecule selected from the group consisting of an antisense molecule, an siRNA, and an shRNA that is complementary to at least a portion of a p75 or p55 TNF-α receptor nucleic acid molecule.

36. The method of claim 35, wherein the inhibitory nucleic acid molecule is selected from the group consisting of an antisense molecule, an siRNA, and an shRNA.

37. (canceled)

38. The method of claim 31, wherein the agent is an antibody or fragment thereof that selectively binds to the p75 or p55 TNF-α receptor.

39-40. (canceled)

41. A pharmaceutical composition for the treatment of a neoplasia, the composition comprising an effective amount of an agent that selectively reduces the expression or activity of a p75 or p55 TNF-α receptor in a neoplastic cell, relative to a reference cell.

42-46. (canceled)

47. The pharmaceutical composition of claim 45, wherein the agent is a sequence selected from the group consisting SEQ ID NO: 1, SEQ ID NO: 2, SEQ ID NO: 3, and SEQ ID NO: 4.

48-52. (canceled)

53. A kit for treating neoplasia comprising an effective amount of an agent that selectively reduces the expression or activity of a p75 or p55 TNF-α receptor in a neoplastic cell and instructions for using the kit to treat neoplasia.

54-66. (canceled)


SEQUENCE LISTING (7 sequences)

SEQ ID NO: 1 - DNA, 21 bp, Artificial Sequence
  Description of Artificial Sequence Synthetic oligonucleotide
ggtggcatctctcttccaatt

SEQ ID NO: 2 - DNA, 21 bp, Artificial Sequence
  Description of Artificial Sequence Synthetic oligonucleotide
ccaaggacactctacgtatct

SEQ ID NO: 3 - DNA, 21 bp, Artificial Sequence
  Description of Artificial Sequence Synthetic oligonucleotide
ggaaccagtttcgtacatgtt

SEQ ID NO: 4 - DNA, 21 bp, Artificial Sequence
  Description of Artificial Sequence Synthetic oligonucleotide
gccaatatgtgaaacatttct

SEQ ID NO: 5 - DNA, 21 bp, Artificial Sequence
  Description of Artificial Sequence Synthetic oligonucleotide
ggaatctcattcgatgcatac

SEQ ID NO: 6 - protein, 461 aa, Homo sapiens
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 7 - protein, 455 aa, Homo sapiens
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR